(19) United States
(12) Patent Application Publication
(10) Pub. No.: US 2025/0143889 A1
(43) Pub. Date: May 8, 2025

(54) LORDOTIC LATERAL IMPLANT, IMPLANT SYSTEM, PROCESS, AND METHODS OF USE

(71) Applicant: Jonathan Melchor, Providence, RI (US)

(72) Inventor: Jonathan Melchor, Providence, RI (US)

(21) Appl. No.: 18/937,086

(22) Filed: Nov. 5, 2024

Related U.S. Application Data
(60) Provisional application No. 63/547,540, filed on Nov. 6, 2023.
(60) Provisional application No. 63/551,180, filed on Feb. 8, 2024.

(51) Int. Cl. A61F 2/44; A61F 2/30

(52) U.S. Cl. CPC A61F 2/447; A61F 2002/30329; A61F 2002/30538

(57) ABSTRACT

A lordotic lateral implant, an implant system, process, and method of use are presented. More specifically, and without limitation, the present disclosure relates an implant, the implant, in example, includes a unique four-wedge design for an improved adjustment system, which includes a left-hand and right-hand threaded shaft option. More specifically, and without limitation, the present disclosure includes ramps for simultaneous, same slope adjustment, which also translates effectively from the shaft when the implant is expanded. Furthermore, but without limitation, this expansion is achieved by only one shaft rotation.

### CROSS-REFERENCE TO RELATED APPLICATIONS

The present application claims priority to the U.S. Provisional Patent Application No. 63/547,540 which was filed on Nov. 6, 2023, which is hereby incorporated by reference herein in its entirety, including any figures, tables, or drawings.

The present application claims priority to the U.S. Provisional Patent Application No. 63/551,180 which was filed on Feb. 8, 2024, which is hereby incorporated by reference herein in its entirety, including any figures, tables, or drawings.

### FIELD OF THE INVENTION

This disclosure relates to a lordotic lateral implant, an implant system, process, and method of use. More specifically, and without limitation, the present disclosure relates an implant, the implant, in example, includes a unique four-wedge design for an improved adjustment system, which includes a left-hand and right-hand threaded shaft option. More specifically, and without limitation, the present disclosure includes ramps for simultaneous, same slope adjustment, which also translates effectively from the shaft when the implant is expanded. Furthermore, but without limitation, this expansion is achieved by only one shaft rotation.

### BACKGROUND OF THE INVENTION

A common and widespread health issue hinders daily life for millions around the globe. Back problems for humans are well known in the art. Back pain is known to plague a great percentage of the population around the globe. Back problems can vary greatly in complexity and type.

Some ongoing pain and discomfort can be solved through stretching. Other problems may need shots and/or ongoing physical therapy. Herniated discs present an even more complex problem which can cause significant pain, along with many other problems for a person experiencing such issues.

More specifically, the human vertebral column, or human spine, is formed of a series of vertebral bodies separated by intervertebral discs. The natural intervertebral disc contains a jelly-like nucleus pulposus surrounded by a fibrous annulus fibrosus. Under an axial load, the nucleus pulposus compresses and radially transfers that load to the annulus fibrosus. The laminated nature of the annulus fibrosus provides it with a high tensile strength and allows it to expand radially in response to the transferred load. If these discs fail or weaken, then the load they are able to transfer, along with a number of other issues, can arise.

Complicating matters further, the discs and spines of humans are not straight lines. Instead, the human spine has a curvature. This normal spine curvature, when viewed from the side, is sometimes referred to as the S-shaped curve. This shape provides an even distribution of weight and flexibility for a human and is thus very important to function and daily comfort.

In some instances, degeneration of the intervertebral disc and/or herniated discs and the like can be treated (at least temporarily) with shots and/or physical therapy and the like. In other instances, a more drastic measure, such as a surgery may be required. Implants, such as those found in the present disclosure, to create a desired spacing of vertebral column bodies with minimal invasiveness don't exist within the state of the art.

Complicating matters further, an easy to install, singularly controlled, implant which creates spacing at the appropriate angle a spine requires does not exist in the art. The S-shape of the spine is very important to shape and comfort. Current implants in the state of the art have attempted to provide angles in the intervertebral disc areas between bodies, however, these implants have not been able to successfully emulate the shape of the curves of a spine, let alone be activated in an effective and easy manner without increasing the invasiveness of an operation. Or worse, some surgeries require fusing of these bodies as a solution to the shape of the spine.

For example, one method of managing these problems is to remove the problematic disc and replace it with a device that restores disc height and allows for bone growth therethrough for the fusion of the adjacent vertebrae. These devices are also referred to as fusion devices.

Thus, there is a long-felt need in the art for a lordotic lateral implant, an implant system, process, and method of use which improves upon the state of the art and solves these problems, provides an implant to replace a disc, while maintaining the shape of a spine, and reducing the impact and/or invasiveness of a surgery through ease of activation is provided herein.

The disclosure herein provides these advantages and others as will become clear from the specification and claims provided.

### SUMMARY OF THE INVENTION

A lordotic lateral implant, an implant system, process, and method of use are presented.

More specifically, and without limitation, the present disclosure relates to a lordotic later implant which includes a unique four-wedge design for an improved adjustment system, which includes a left-hand and right-hand threaded shaft option. More specifically, and without limitation, the present disclosure includes ramps for simultaneous, same slope adjustment, which also translates effectively from the shaft when the implant is expanded. Furthermore, but without limitation, this expansion is achieved by only one shaft rotation.

Thus, it is a primary object of the disclosure to provide a lordotic lateral implant, an implant system, process, and method of use that improves upon the state of the art. Thus, there is a long-felt need in the art for a lordotic lateral implant, an implant system, process, and method of use which improves upon the state of the art and solves these problems, provides an implant to replace a disc, while maintaining the shape of a spine, and reducing the impact and/or invasiveness of a surgery through ease of activation is provided herein.

Another object of the disclosure is to provide a lordotic lateral implant, an implant system, process, and method of use that provides a single shaft which includes both left-handed threading and right-handed threading.

Yet another object of the disclosure is to provide a lordotic lateral implant, an implant system, process, and method of use that utilizes a four wedge design.

Another object of the disclosure is to provide a lordotic lateral implant, an implant system, process, and method of use that includes wedges which are symmetrical in two planes.

Yet another object of the disclosure is to provide a lordotic lateral implant, an implant system, process, and method of use that utilizes ramps on both upper and lower plates.

Another object of the disclosure is to provide a lordotic lateral implant, an implant system, process, and method of use that utilizes two ramps with the same slope.

Yet another object of the disclosure is to provide a lordotic lateral implant, an implant system, process, and method of use that provides for a shaft which translates when the implant is expanded.

Another object of the disclosure is to provide a lordotic lateral implant, an implant system, process, and method of use that provides an expansion and lordotic angle by rotating only a single shaft.

Yet another object of the disclosure is to provide a lordotic lateral implant, an implant system, process, and method of use that provides multiple wedges with linear ramps.

Another object of the disclosure is to provide a lordotic lateral implant, an implant system, process, and method of use that includes wedges which maintain contact with the endplate ramps at all times.

Yet another object of the disclosure is to provide a lordotic lateral implant, an implant system, process, and method of use that includes outer wedges which rotate with respect to inner wedges along the shaft axis.

Another object of the disclosure is to provide a lordotic lateral implant, an implant system, process, and method of use that accomplishes lordotic expansion with the rotation of one shaft.

Yet another object of the disclosure is to provide a lordotic lateral implant, an implant system, process, and method of use that provides using a single shaft to expand the height of both sides of the implant, simultaneously.

Another object of the disclosure is to provide a lordotic lateral implant, an implant system, process, and method of use that provides a shaft which is not fixed to either the upper or the lower plates.

Yet another object of the disclosure is to provide a lordotic lateral implant, an implant system, process, and method of use that includes an over expansion feature.

Another object of the disclosure is to provide a lordotic lateral implant, an implant system, process, and method of use that includes an anti-collapse feature.

Yet another object of the disclosure is to provide a lordotic lateral implant, an implant system, process, and method of use that includes an anti-collapse lock.

Another object of the disclosure is to provide a lordotic lateral implant, an implant system, process, and method of use that requires less invasiveness with respect to relatively related operations.

Yet another object of the disclosure is to provide a lordotic lateral implant, an implant system, process, and method of use that improves upon the shape of the spine of a user, compared to other implants.

Another object of the disclosure is to provide a lordotic lateral implant, an implant system, process, and method of use that is easy to use.

Yet another object of the disclosure is to provide a lordotic lateral implant, an implant system, process, and method of use that is safe to use.

Another object of the disclosure is to provide a lordotic lateral implant, an implant system, process, and method of use that is accurate.

Yet another object of the disclosure is to provide a lordotic lateral implant, an implant system, process, and method of use that is quick and efficient.

Yet another object of the disclosure is to provide a lordotic lateral implant, an implant system, process, and method of use that is robust.

Another object of the disclosure is to provide a lordotic lateral implant, an implant system, process, and method of use that saves time for a user.

Another object of the disclosure is to provide a lordotic lateral implant, an implant system, process, and method of use that are high quality.

Yet another object of the disclosure is to provide a lordotic lateral implant, an implant system, process, and method of use that improves comfort for a user.

These and other objects, features, or advantages of the present disclosure will become apparent from the specification and claims.

### BRIEF DESCRIPTION OF DRAWINGS

The drawings accompanying and forming part of this specification are included to depict certain aspects of the disclosure.

FIG. 1 is a perspective view of the lordotic implant; the view showing an upper plate engaged with a lower plate; the view showing a shaft, partially, located in operational position within the lordotic implant.

FIG. 2 is a perspective view of the lordotic implant; the view showing an upper plate engaged with a lower plate; the view showing a shaft, partially, located in operational position within the lordotic implant.

FIG. 3 is a perspective view of the lordotic implant; the view showing an upper plate engaged with a lower plate; the view showing a shaft, partially, located in operational position within the lordotic implant.

FIG. 4 is a perspective view of the lordotic implant; the view showing an upper plate engaged with a lower plate; the view showing a shaft, partially, located in operational position within the lordotic implant.

FIG. 5 is an exploded view showing a lordotic implant; the view showing an upper plate; the view showing a lower plate; the view showing a shaft; the view showing a plurality of stops; the view showing an outer proximal wedge; the view showing an inner proximal wedge; the view showing an outer distal wedge; the view showing an inner distal wedge.

FIG. 6 is an exploded view showing a lordotic implant; the view showing an upper plate; the view showing a lower plate; the view showing a shaft; the view showing a plurality of stops; the view showing an outer proximal wedge; the view showing an inner proximal wedge; the view showing an outer distal wedge; the view showing an inner distal wedge.

FIG. 7 is an exploded view showing a lordotic implant; the view showing an upper plate; the view showing a lower plate; the view showing a shaft; the view showing a plurality of stops; the view showing an outer proximal wedge; the view showing an inner proximal wedge; the view showing an outer distal wedge; the view showing an inner distal wedge.

FIG. 8 is an exploded view showing a lordotic implant; the view showing an upper plate; the view showing a lower plate; the view showing a shaft; the view showing a plurality of stops; the view showing an outer proximal wedge; the view showing an inner proximal wedge; the view showing an outer distal wedge; the view showing an inner distal wedge.

FIG. 9 is an exploded view showing a lordotic implant; the view showing an upper plate; the view showing a lower plate; the view showing a shaft; the view showing a plurality of stops; the view showing an outer proximal wedge; the view showing an inner proximal wedge; the view showing an outer distal wedge; the view showing an inner distal wedge.

FIG. 10 is a perspective view showing a lower plate; the view showing the exterior of the lower plate.

FIG. 11 is a perspective view showing a lower plate; the view showing the interior of the lower plate.

FIG. 12 is a perspective view showing an upper plate; the view showing the interior of the upper plate.

FIG. 13 is a perspective view showing an upper plate; the view showing the exterior of the upper plate.

FIG. 14 is a perspective view of an inner proximal wedge.

FIG. 15 is a perspective view of an outer proximal wedge.

FIG. 16 is a perspective view of an outer distal wedge.

FIG. 17 is a perspective view of an inner distal wedge.

FIG. 18 is a perspective view of an inner distal wedge.

FIG. 19 is a perspective view of an outer distal wedge.

FIG. 20 is a perspective view of an outer proximal wedge.

FIG. 21 is a perspective view of an inner proximal wedge.

FIG. 22 is a perspective view of a secondary feature.

FIG. 23 is a perspective view of a secondary feature.

FIG. 24 is a perspective view of a stop.

FIG. 25 is a perspective view of a shaft.

FIG. 26 is a perspective view of a shaft.

FIG. 27 is a perspective view of the lordotic implant; the view showing an upper plate engaged with a lower plate; the view showing a shaft, partially, located in operational position within the lordotic implant; the view showing a lock/unlock feature.

FIG. 28 is a cross-sectional view of the lordotic implant; the view showing an upper plate engaged with a lower plate; the view showing a shaft, partially, located in operational position within the lordotic implant.

FIG. 29 is a cross-sectional view of the lordotic implant; the view showing an upper plate engaged with a lower plate; the view showing a shaft, partially, located in operational position within the lordotic implant.

FIG. 30 is a cross-sectional view of the lordotic implant; the view showing an upper plate engaged with a lower plate; the view showing a shaft, partially, located in operational position within the lordotic implant.

FIG. 31 is a side view of the lordotic implant; the view showing an upper plate engaged with a lower plate; the view showing a shaft, partially, located in operational position within the lordotic implant.

FIG. 32 is a side view of the lordotic implant; the view showing an upper plate engaged with a lower plate; the view showing a shaft, partially, located in operational position within the lordotic implant.

FIG. 33 is a side view of the lordotic implant; the view showing an upper plate engaged with a lower plate; the view showing a shaft, partially, located in operational position within the lordotic implant.

FIG. 34 is a side view of the lordotic implant; the view showing an upper plate engaged with a lower plate; the view showing a shaft, partially, located in operational position within the lordotic implant; the view showing an indicator feature.

### DESCRIPTION

In the following detailed description, reference is made to the accompanying drawings which form a part hereof, and in which is shown by way of illustration specific embodiments in which the disclosure may be practiced. These embodiments are described in sufficient detail to enable those skilled in the art to practice the disclosure, and it is to be understood that other embodiments may be utilized and that mechanical, procedural, and other changes may be made without departing from the spirit and scope of the disclosure(s).

The following detailed description is, therefore, not to be taken in a limiting sense, and the scope of the disclosure(s) is defined only by the appended claims, along with the full scope of equivalents to which such claims are entitled.

As used herein, the terminology such as vertical, horizontal, top, bottom, front, back, end, sides and the like are referenced according to the views, pieces and figures presented. It should be understood, however, that the terms are used only for purposes of description, and are not intended to be used as limitations. Accordingly, orientation of an object or a combination of objects may change without departing from the scope of the disclosure.

Reference throughout this specification to “one embodiment,” “an embodiment,” “one example,” or “an example” means that a particular feature, structure, or characteristic described in connection with the embodiment or example is included in at least one embodiment of the present disclosure. Thus, the appearance of the phrases “in one embodiment,” “in an embodiment,” “one example,” or “an example” in various places throughout this specification are not necessarily all referring to the same embodiment or example.

Furthermore, the particular features, structures, databases, or characteristics may be combined in any suitable combinations and/or sub-combinations in one or more embodiments or examples. In addition, it should be appreciated that the figures provided herewith are for explanation purposes to persons ordinarily skilled in the art and that the drawings are not necessarily drawn to scale.

### System:

With reference to the figures, a lordotic lateral implant, an implant system, process, and method of use 100 are presented. a lordotic lateral implant, an implant system, process, and method of use 100 (hereafter referred to as “lordotic system”, “implant system”, “lordotic implant system”, simply “implant”, or simply “system”) is formed of any suitable size, shape and design.

In the arrangement shown, as one example, system 100 includes a lower plate 120, an upper plate 140, and a shaft 180, an outer proximal wedge 210, an inner proximal wedge 220, an outer distal wedge 230, and an inner distal wedge 240, among other features, components, and functionality.

In the arrangement shown, as one example, system 100 is configured to provide lordotic expansion while maintaining the connection of expansion. Furthermore, system 100 is configured to provide an expandable spacer which expands both opposing sides of the spacer utilizing a single shaft. Furthermore, implant 100 provides this expansion simultaneously. Meaning the system can provide both upper and lower expansion at the same time. Furthermore, system 100 is configured to provide a single shaft with both left-handed threading and right-handed threading which offer a number of advantages in the art. In the arrangement shown, as one example, system 100 is configured to provide up to 15 degrees of lordotic adjustment. This 15 degrees is not necessarily from 0 degrees but will vary depending on need. This may be 5 degrees to 20 degrees. This may also be 10 degrees to 25 degrees. This may constitute a 10 mm expansion. Furthermore, up to 5 degrees is hereby contemplated; up to 10 degrees is hereby contemplated; up to 20 degrees is hereby contemplated; up to 25 degrees is hereby contemplated; up to 30 degrees is hereby contemplated; up to 35 degrees is hereby contemplated. Similarly, this range may be incremental such as providing 1 to 5 degrees, 1 to 10 degrees, 1 to 15 degrees, 1 to 20 degrees, 1 to 25 degrees, 1 to 30 degrees, and the like.

Furthermore, system 100 is configured with a unique four-wedge design which provides for expansion of the spacer on both sides which provides an optimal and/or customized angle of expansion. Furthermore, system 100 is configured to provide wedges which are symmetrical in two planes. Furthermore, system 100 is configured to provide ramps on both the lower plate 120 and the upper plate 140 which have the same slopes. Furthermore, system 100 is configured to provide a shaft which translates when the implant is expanded. Said another way, when the shaft is rotated on its axis, both plates, the upper and lower plate, expand at equal rates and/or expand equally in the amount of spatial volume created between the plate and the shaft. Said another way, if a lower plate is steady and/or stabilized, then the shaft translates and the upper plate rotates.

Furthermore, system 100 is configured to provide expansion of the spacer and lordotic angle, achieved by the rotation of only a single shaft. This provides for a minimized invasiveness during installation and/or removal of the implant. In this way, only a single tool and single expansion is required. Compared to the current state of the art requiring multiple expansions and adjustments and/or relocations of an expansion tool.

In the arrangement shown, as one example, system 100 is configured with a number of unique features which provide unique functionality not achieved or considered in the state of the art. System 100 is configured to provide wedges which have linear ramps. Furthermore, system 100 is configured with wedges which are in constant contact with the lower plate and/or upper plate ramps, at all times, for smooth operation. These benefits, in association with the spacing benefits and other advantages discussed herein provide for a unique lordotic implant and method of installation of the implant and/or removal of the implant and/or operation of the implant when the implant is in use.

Furthermore, system 100 is configured to provide outer wedges (an outer proximal wedge and an outer distal wedge; to be further described herein) which translate and/or operate with respect to inner wedges (an inner proximal wedge and an inner distal wedge; to be further described herein) along the shaft axis. Said another way, the wedges force the upper plate and the lower plate to expand and/or contract when the shaft rotates. This process is further described herein.

This unique design offers a number of advantages, such as use of a single shaft to provide expansion of both opposing sides (upper plate and lower plate) of the spacer or implant simultaneously and at a desired ramp angle. Ramp angle is critical for success. Furthermore, and said another way, system 100 is configured to provide lordotic expansion with the rotation of a single shaft and/or installation through the rotation of a single shaft from a single end of that shaft.

Another feature of system 100, is that system 100 is configured with a shaft which is not fixed. Said another way, the shaft 180 (to be further described herein) is not fixed to either the upper plate or lower plate, providing further advantages and benefits in the art. This development and extensively designed feature of the shaft 180 (being a unique floating shaft) is hugely advantageous over the state of the art. The current state of the art teaches two shafts attached to each opposing side for moving the two sides separately to achieve a goal (such as a desired angle and expansion). Current systems therefore require multiple adjustments and a much more invasive installation and/or removal.

Furthermore, and said another way, system 100 is configured to provide a consistent height of expansion on both sides of the implant while expansion occurs. Said another way, system 100 is configured to provide uniform expansion from a single adjustment. This uniform expansion may be adjusted or varied depending on the needs of a patient. Typically a uniform expansion involves reaching a desired uniform angle. In some cases, this may be different angles and this expansion is hereby contemplated and considered.

Furthermore, system 100 is configured to provide an over expansion feature.

Furthermore, system 100 is configured to provide the state of the art with an anti-collapse feature.

These and other benefits and advantages will become more apparent with the description herein and the accompanying figures.

### Lower Plate:

In the arrangement shown, as one example, system 100 includes a lower plate 120 and an upper plate 140 adjustably situated and/or operably coupled to the lower plate 120.

In the arrangement shown, as one example, the lower plate 120 is roughly rectangular in shape with soft edges, extending a length from a first end 122 to a second end 123 between opposing sides 124.

Lower plate 120 includes a plurality of curved ribs 126. The plurality of curved ribs 126 provide for an interconnection and stability between the lower plate and the upper plate. Said another way, the plurality of curved ribs 126 are configured to provide guidance to the movement and interactions of the upper plate 140 and the lower plate 120. In the arrangement shown, as one example, the plurality of curved ribs 126 are concentric. Furthermore, the plurality of ribs 126 include a combination of ribs and grooves to provide an interconnection and stability between the lower plate and the upper plate. Said another way, the movement and/or interaction motion of the upper plate 140 respective to the lower plate 120 is non-linear (in one example). This provides a number of benefits, including but not limited to, expanding and/or contracting the implant at an angular or curved angular motion. Linear movement is also hereby contemplated for use.

In the arrangement shown, as one example, the lower plate 120 also includes a curved center post 128 or center post 128. Curved center post 128 or center post 128 is formed of any suitable size, shape and is designed and configured to receive shaft 180. Said another way, the longitudinal displacement and/or any other displacement of the shaft 180 is restricted by the curved center post 128 or plurality of curved center posts 128. Said another way, center post 128 is curved on the outside but linear on the inside. In other words, the center post is straight or forms a straight line on the interior side of the center post 128 but is curved on the exterior side of the center post 128.

The center posts or plurality of center posts 128 are configured to interact with the first guide 194 and second guide 196 of the shaft (to be further described herein). The combination of the plurality of center posts 128, interlocking with the first guide 194 and second guide 196 of the shaft provide for prevention of movement, other than rotation of the shaft along the shafts center axis. Furthermore, the shaft travels along the curved center post 128 while the implant is expanded and/or contracted. This plurality of center posts 128 aligned at the same angle of the desired expansion to allow for a smooth movement of the shaft during expansion and/or contraction. Furthermore, the center post has a plurality of ridges 197 for engages the guides of the shaft. Furthermore, and in the arrangement shown as one example, center post 128 are all concentric. Furthermore, the plurality of center posts 128 include a combination of ribs and grooves to provide an interconnection and stability between the lower plate and the upper plate.

In the arrangement shown, as one example, a first guide 194 and second guide 196 are shown. Two guides, one guide, three guides, four guides, five guides, or more guides are hereby contemplated for use.

### Ramps of Lower Plate:

First Ramp: In the arrangement shown, as one example, lower plate 120 includes a first ramp 130. First ramp 130 of the lower plate is configured to operate with and/or provide a smooth sliding surface for either the inner proximal wedge or the inner distal wedge. The ramp provides a channel for the wedge to slide along. Said another way, when the shaft is rotated, causing the wedge to move, the wedge moves and/or slides along the channel of the ramp.

Second Ramp: In the arrangement shown, as one example, system 100 includes a second ramp 131. Second ramp 131 of the lower plate is configured to operate with and/or provide a smooth sliding surface for either the inner proximal wedge or the inner distal wedge. The ramp provides a channel for the wedge to slide along. Said another way, when the shaft is rotated, causing the wedge to move, the wedge moves and/or slides along the channel of the ramp.

In the arrangement shown, as one example, lower plate 120 includes a first ramp 130 and a second ramp 131. However, lower plate 120 may include additional ramps. A third ramp, fourth ramp, fifth ramp, sixth ramp, or more ramps are hereby contemplated for use along with a coinciding or varying number of wedges.

In the arrangement shown, as one example, lower plate 120 includes a plurality of protrusions 133. Plurality of protrusions 133 are formed of any suitable size, shape, and design and are configured to provide grip-like functionality when engaged. In the arrangement shown, as one example, the plurality of protrusions 133 are each pyramid like in shape so as to provide a shallow pointed engagement. Other shapes for the plurality of protrusions 133 are hereby contemplated for use such as ridges, alternative materials, and the like.

In the arrangement shown, as one example, lower plate 120 includes at least one lumen 134. Lumen 134 is intended to provide for a bone graft. At least one lumen is included herein as an example. However, other numbers are lumens are hereby contemplated for use such as zero lumens, two lumens, three lumens, or the like.

In the arrangement shown, as one example, lower plate 120 also includes a plurality of attachment features 135, an interior surface 136, and an exterior surface 138, among other features, components, and functionality. Plurality of attachment features 135 are all concentric, in the arrangement shown, as one example. Furthermore, plurality of attachment features 135 include a combination of ribs and grooves to provide an interconnection and stability between the lower plate and the upper plate.

### Upper Plate:

In the arrangement shown, as one example, system 100 includes an upper plate 140 and a lower plate 120 adjustably situated and/or operably coupled.

In the arrangement shown, as one example, the upper plate 140 is roughly rectangular in shape with soft edges, extending a length from a first end 142 to a second end 143 between opposing sides 144.

Upper plate 140 includes a plurality of curved rib apertures 146. The plurality of curved rib apertures 146 provide for an interconnection and stability between the lower plate and the upper plate. Said another way, the plurality of curved rib apertures 146 are configured to provide guidance to the movement and interactions of the upper plate 140 and the lower plate 120. In the arrangement shown, as one example, the plurality of curved rib apertures 146 are concentric. Additionally, the plurality of curved rib apertures 146 may also include ribs or a plurality of ribs. Said another way, the movement and/or interaction motion of the upper plate 140 respective to the lower plate 120 is non-linear (in one example). This provides a number of benefits, including but not limited to, expanding and/or contracting the implant at an angular or curved angular motion. Linear movement is also hereby contemplated for use. Furthermore, and in the arrangement shown as one example, the plurality of curved rib apertures 146 are all concentric. Furthermore, the plurality of curved rib apertures 146 include a combination of ribs and grooves to provide an interconnection and stability between the lower plate and the upper plate.

In the arrangement shown, as one example, the upper plate 140 also includes a curved center post aperture 148 or center post aperture 148. Curved center post aperture 148 or center post aperture 148 is formed of any suitable size, shape and is designed and configured to receive the curved center post 128 of the lower plate 120 and thus the shaft 180. Said another way, the center post aperture 148, in combination with the center post 128 provides for the prevention of displacement of the shaft 180. Furthermore, and in the arrangement shown as one example, the center post apertures 148 are all concentric. Furthermore, the plurality center post apertures 148 include a combination of ribs and grooves to provide an interconnection and stability between the lower plate and the upper plate.

The center post apertures or plurality of center post apertures 148 are configured to interact with the first guide 194 and second guide 196 of the shaft (to be further described herein). The combination of the plurality of center posts 128 and the center post apertures 148, interlocking with the first guide 194 and second guide 196 of the shaft provide for prevention of movement, other than rotation of the shaft along the shafts center axis. Furthermore, the shaft travels along the curved center post 128 while the implant is expanded and/or contracted. This plurality of center posts 128 aligned at the same angle of the desired expansion to allow for a smooth movement of the shaft during expansion and/or contraction. In the arrangement shown, as one example, a first guide 194 and second guide 196 are shown. Two guides, one guide, three guides, four guides, five guides, or more guides are hereby contemplated for use. Furthermore, and said another way, center post is configured to prevent the shaft from translating in the direction along the length of the shaft. In the arrangement shown, center post includes a ridge which is between the first guide and the second guide, preventing this type of movement or translation. Similarly, center post is configured with a plurality of center posts to prevent movement is a perpendicular direction.

### Ramps of Upper Plate:

First Ramp: In the arrangement shown, as one example, upper plate 140 includes a first ramp 150. First ramp 150 of the upper plate is configured to operate with and/or provide a smooth sliding surface for either the outer proximal wedge or the outer distal wedge. The ramp provides a channel for the wedge to slide along. Said another way, when the shaft is rotated, causing the wedge to move, the wedge moves and/or slides along the channel of the ramp. In the arrangement shown, as one example, the wedges are uniquely formed in a “T” shape which causes the endplates to expand and/or contract when being utilized.

Second Ramp: In the arrangement shown, as one example, upper plate 140 includes a second ramp 152. Second ramp 152 of the upper plate is configured to operate with and/or provide a smooth sliding surface for either the outer proximal wedge or the outer distal wedge. The ramp provides a channel for the wedge to slide along. Said another way, when the shaft is rotated, causing the wedge to move, the wedge moves and/or slides along the channel of the ramp. In the arrangement shown, as one example, the wedges are uniquely formed in a “T” shape which causes the endplates to expand and/or contract when being utilized.

In the arrangement shown, as one example, upper plate 140 includes a first ramp 150 and a second ramp 152. However, upper plate 140 may include additional ramps. A third ramp, fourth ramp, fifth ramp, sixth ramp, or more ramps are hereby contemplated for use along with a coinciding or varying number of wedges.

Stops: In the arrangement shown, as one example, upper plate 140 includes a plurality of stops 151/153, or a first stop 151 and a second stop 153. The first stop and/or second stop prevents the implant from moving beyond are particular desired expansion and/or contraction. Two stops are shown in the example provided herein. However, other numbers of stops are hereby contemplated for use such as no stops, one stop, two stops, three stops, four stops, five stops, or more. Furthermore, system 10 includes a plurality of stop apertures 154/156 for accepting the plurality of stops 151/153.

In the arrangement shown, as one example, upper plate 140 includes a plurality of protrusions 164. Plurality of protrusions 164 are formed of any suitable size, shape, and design and are configured to provide grip-like functionality when engaged. In the arrangement shown, as one example, the plurality of protrusions 164 are each pyramid like in shape so as to provide a shallow pointed engagement. Other shapes for the plurality of protrusions 164 are hereby contemplated for use such as ridges, alternative materials, and the like.

In the arrangement shown, as one example, upper plate 140 includes at least one lumen 166. Lumen 166 is intended to provide for a bone graft. At least one lumen is included herein as an example. However, other numbers are lumens are hereby contemplated for use such as zero lumens, two lumens, three lumens, or the like.

In the arrangement shown, as one example, upper plate 140 also includes a plurality of attachment features 168, an interior surface 170, and an exterior surface 172, among other features, components, and functionality.

### Shaft:

Implant 100 includes a shaft 180. Shaft 180 is formed of any suitable size, shape and design and is configured to provide expansion and contraction of the implant 100. Shaft 180 extends a length from a first end 184 to a second end 186. Shaft 180 includes an outer surface 188 which may be all or partially threaded.

In the arrangement shown, as one example, shaft 180 includes a first threading 190 and a second threading 192. The first threading 190, in one example, is a right-hand thread or right-handed threading, while the second threading 192 is a left hand thread or left handed threading. Other threading combinations and the like are hereby contemplated for use. Shaft 180 may be referred to as “a single shaft”, “singular shaft”, or “single shaft”.

Furthermore, the right hand thread 190 (or “first threading”) may be on one side of the shaft (nearer a first end) and the left hand thread 192 (or “second threading”) may be on another side of the shaft (nearer a second end). In this way, the longitudinal movement of the shaft 180 is restricted and/or provided limitations. The opposing threading provides for dual movement of all wedges simultaneously to provide for equalized expansion at the desired angle.

Said another way, the opposing threading forces the wedges to slide in opposing directions along the ramps such that the first end of the implant expands and/or contracts at an equal rate of distance to the second end. Said another way, the first end expands or grows in volume at an equal angle and distance as the second end via movement of a single shaft 180 and/or rotation of a single shaft 180.

In the arrangement shown, as one example, shaft also includes a first guide 194 and a second guide 196.

Furthermore, in the arrangement shown, as shown in one example, shaft 180 includes a cavity 198. In one arrangement, the cavity or aperture adjustment 198 is configured on the proximal side so as to allow engagement with a tool. The engagement with the tool can cause rotation to the shaft. This tool may be a uniquely configured and shaped tool. Cavity may also be configured to interact with known tooling such as a torx tool, allen type tool (allen wrench), philips shape, and the like.

Furthermore, and in the arrangement shown, as one example, the shaft displaces longitudinally. The longitudinal displacement of the shaft is restricted by this curved center post 128. The shaft travels along the curved center post 128 while the implant is expanded and/or contracted.

### Wedges:

In the arrangement shown, as one example, system 100 also includes an outer proximal wedge, an outer distal wedge, an inner proximal wedge, and an outer proximal wedge.

In the arrangement shown, as one example, the plurality of wedges are configured in concert with the upper and lower plates such that the wedges are received on the shaft and are movable along the shaft so as to adjust the position of the upper plate and the lower plate. See figures for further illustrations on this operation and movement.

In the arrangement shown, as one example, the outer proximal wedge includes a linear ramp. Similarly, the outer distal wedge includes a linear ramp. The linear ramp of the outer proximal wedge and the linear ramp of the outer distal wedge each have a threaded hole which engages directly with the thread of the shaft.

In this way, when the shaft is rotated in an opening direction, the wedges move toward each other, causing the linear ramp to slide against the linear ramp, this causes lordotic expansion. Furthermore, when the shaft is rotated in a closing direction, the wedges move away from one another, causing the linear ramp to slide away from or along the linear ramp in a disengaging fashion, which causes lordotic contraction of the implant.

In the arrangement shown, as one example, the inner proximal wedge and the inner distal wedge may be threaded. An unthreaded inner proximal wedge and an unthreaded inner distal wedge are also hereby contemplated for use. Inner wedges move along the shaft.

In the arrangement shown, as one example, outer proximal wedge and outer distal wedge each include linear ramps which engage the upper plate. The inner proximal wedge and the inner distal wedge have linear ramps which engage the lower plate.

Said another way, when the shaft is rotated to expand and/or engage the implant 100, the outer proximal wedge and the outer distal wedge move and/or slide and/or engage the ramps in a frictional fashion, which forces the upper plate to expand lordotically in a direction away from the lower plate. Similarly, when the shaft is rotate to expand the implant, the inner proximal wedge and the inner distal wedge ride along, slide along, and or frictionally force the ramps of the lower plate. In this way, the outer and inner wedges move in the opposite direction relative to each other on the shaft. Furthermore, when the implant is expanded and/or in the expanded position, the height on one side and/or the distance from the lower plate to the upper plate on one of the opposing sides is larger than the distance from the upper plate to the lower plate of the second of the opposing sides and/or of the opposite side of the opposing sides. In this way, the implant acts uniquely as a lordotic implant and provides simultaneous expansion using a single shaft but creating different distances on the opposing sides of the implant. In the arrangement shown, as one example, the wedges are uniquely formed in a “T” shape which causes the endplates to expand and/or contract when being utilized.

### Set Screw and Hole:

In the arrangement shown, as one example, system 100 includes a set screw 260. Set screw 260 is formed of any suitable size, shape, and design and is configured to prevent the implant from collapsing by engaging. The set screw 260 stops the translation of one of the internal wedges when engaged. In this way, the set screw 260, when in an engaged position, prevents expansion or contraction of the lordotic implant. The set screw 260 allows for the translation of at least one of the internal wedges when disengaged. In this way, the set screw 260 maintains an expanded implant.

The set screw 260 is located in a threaded hole in the lower plate and or the upper plate. The set screw 260 is configured with a threading which interacts with the threading of an aperture of the upper plate and/or lower plate such that the set screw can be tightened, via movement inward against or in the path of movement of the wedges and/or loosened, via movement outward or away from the interior and/or path of movement of the wedges.

Furthermore, the plate, the upper plate and/or the lower plate, which holds the set screw 260 also includes a hole perpendicular to the screw hole. In this way, when the set screw 260 is in an engaged position, the hole is covered. Said another way, the lock/unlock indicator feature 280 is filled. In this way, the hole (being covered) conveys to the surgeon or other user, under fluoro, that the implant is locked and secured in place. If the hole is visible to the surgeon, or the hole is empty, then the surgeon has not locked the implant into place. In this way, the lock/unlock indicator feature 280 displays and provides clear indication as to whether the implant is locked and/or unlocked. Said another way, a lock and/or unlock visibility feature 280 is contemplated. Lock and/or unlock visibility feature 280 provides an indicator that the system is locked in place and/or unlocked (allowing for expansion and/or contraction). Lock and/or unlock visibility feature 280 may also be referred to as indicator feature 280, visibility feature 280, visibility indicator feature 280, or lock/unlock indicatore feature 280.

### Over Expansion and Other Features:

Over expansion and/or a particular distance of expansion can be predetermined prior to installation of the implant. System is configured to prevent over expansion with the inclusion of a sleeve around the shaft which stops expansion at a particular preset, depending on the needs of the patient. In another way, a stop can be installed which prevents the expansion past a particular distance. This can be achieved by inclusion of a stop on the upper plate.

In an alternative embodiment, and after an implant has been installed, the stabilization of that implant is important. For longevity and effectiveness, implant includes a locking feature of the shaft (locking in concert with the outer proximal wedge) utilizing a secondary feature so as to lock the implant at the desired expansion level.

In the arrangement shown, as one example, the upper plate 140 and lower plate 120 include ramps. Ramps are configured on the distal side. These ramps are configured to allow easier insertion and less invasive implanting into the vertebral disc space. Similarly, and said another way, the outer proximal wedge includes and is configured to have a boss on the proximal side which provides for engagement with an inserter. This aids in insertion of the implant. This also causes installation to be less invasive than current methods in the state of the art. Additionally, implant 100 includes a plurality of protrusions which provide for improved engagement with the end plates of a vertebrae and/or bone surface and/or engagement surface. Furthermore, and in the arrangement shown, system 100 includes a lower plate and an upper plate each having a lumen which is configured to accept a bone graft.

### Another Embodiment

In another arrangement, as shown herein, system may include a hand tool, an installation tool, a plurality of attachment features, a user, a patient, a doctor, a medical professional, a spine, a plurality of bones or bodies, and an implant.

### Alternative Embodiment

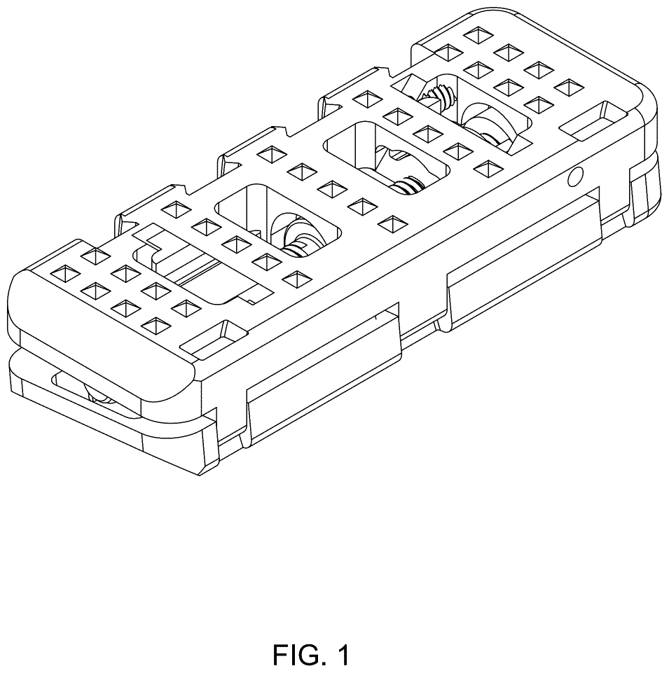

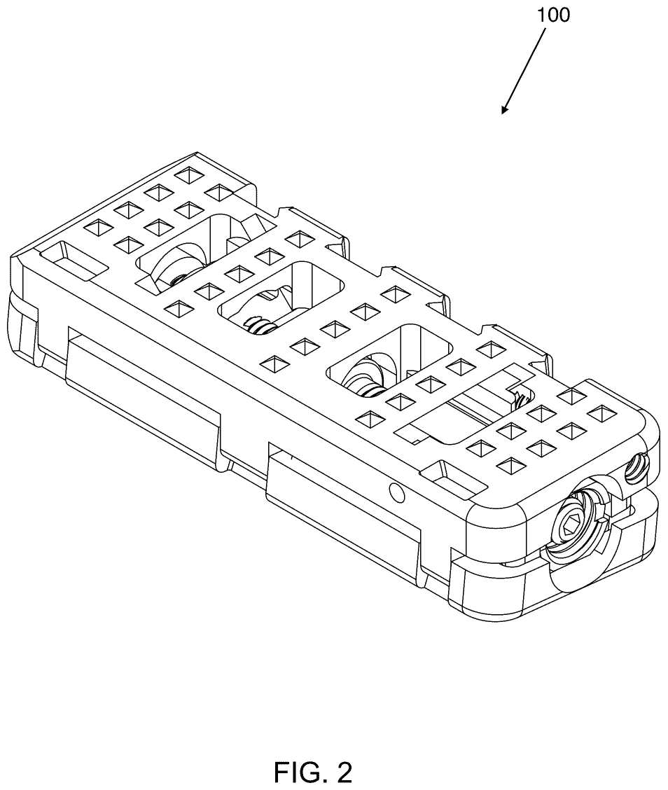

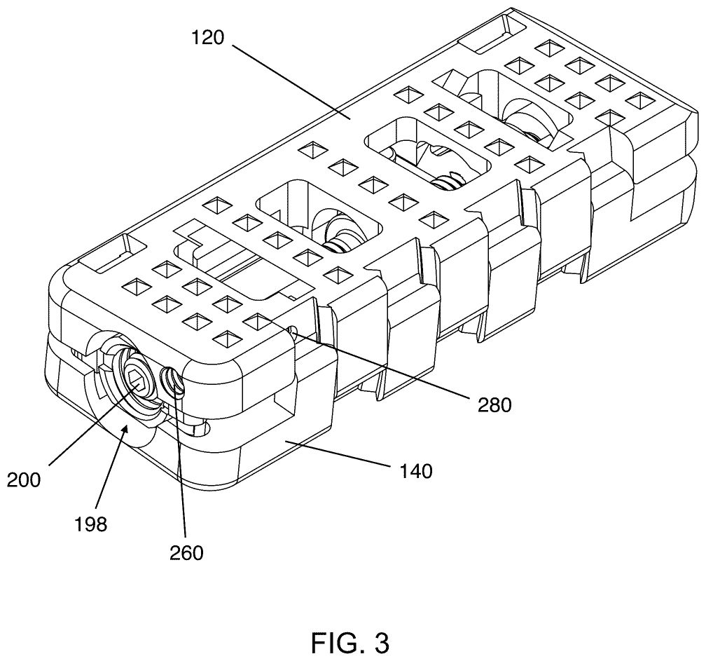

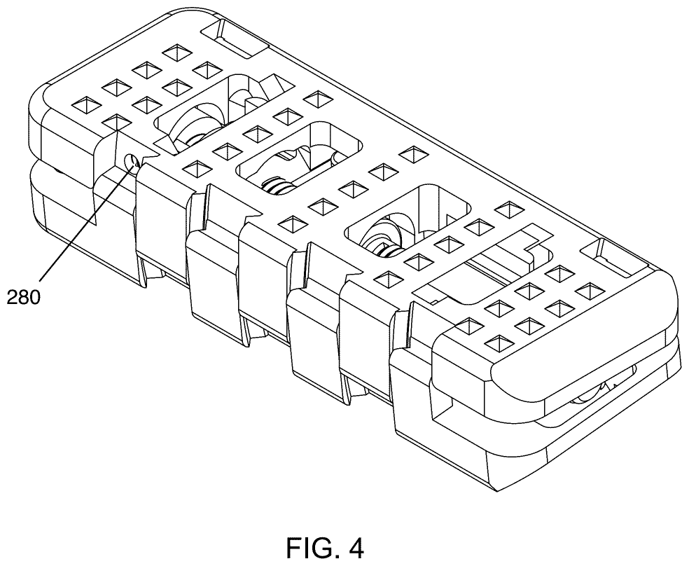

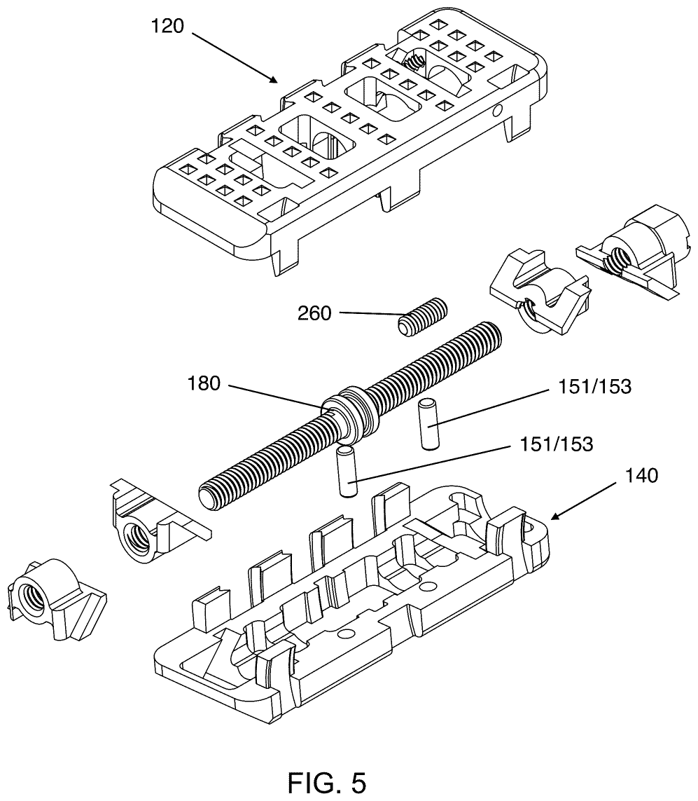

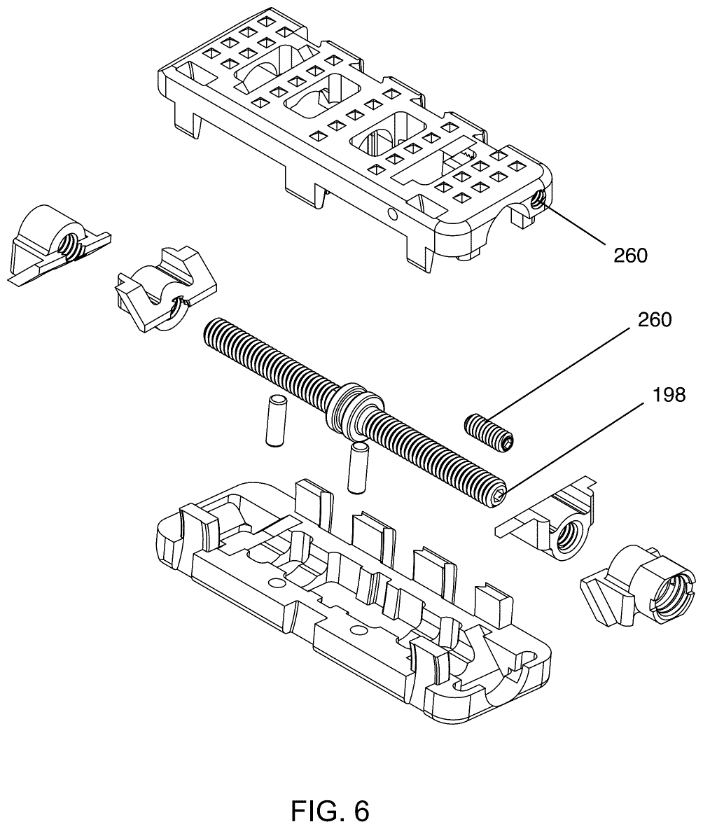

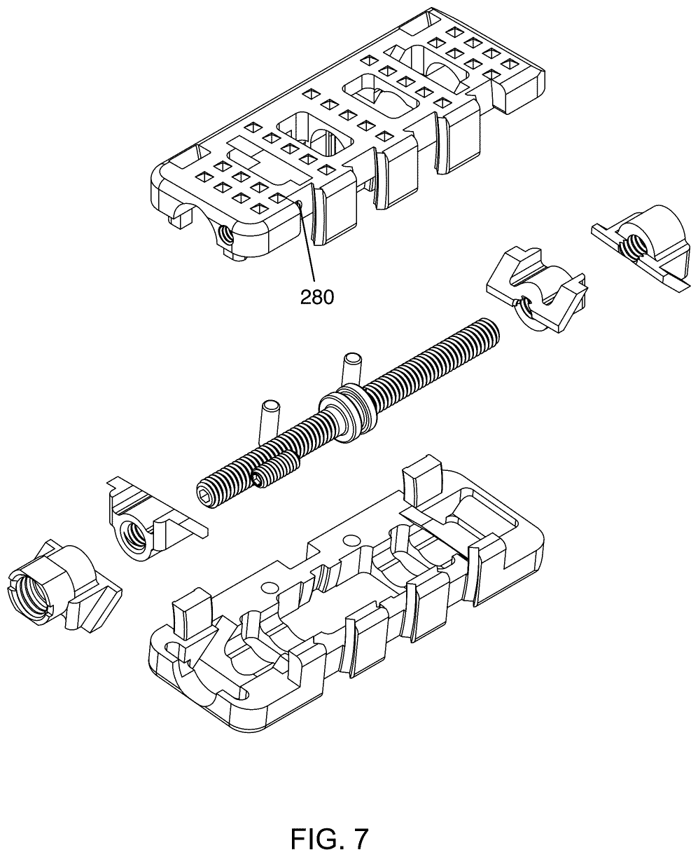

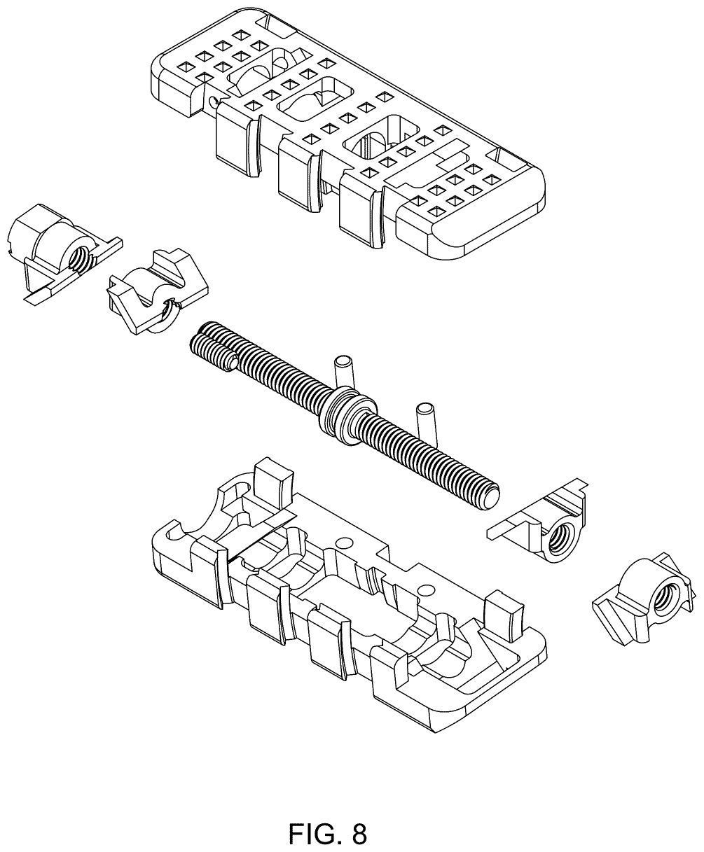

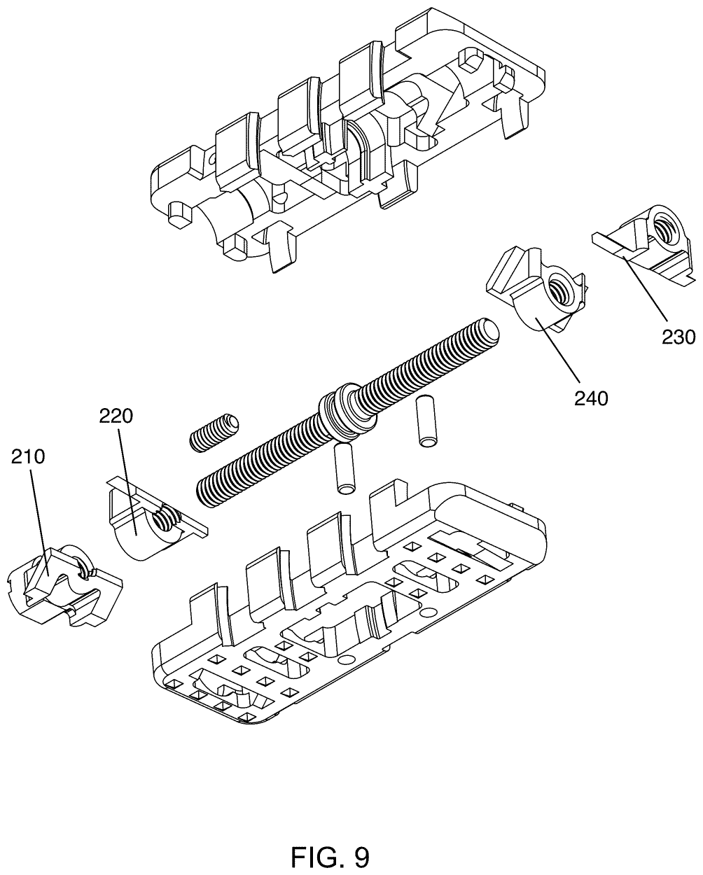

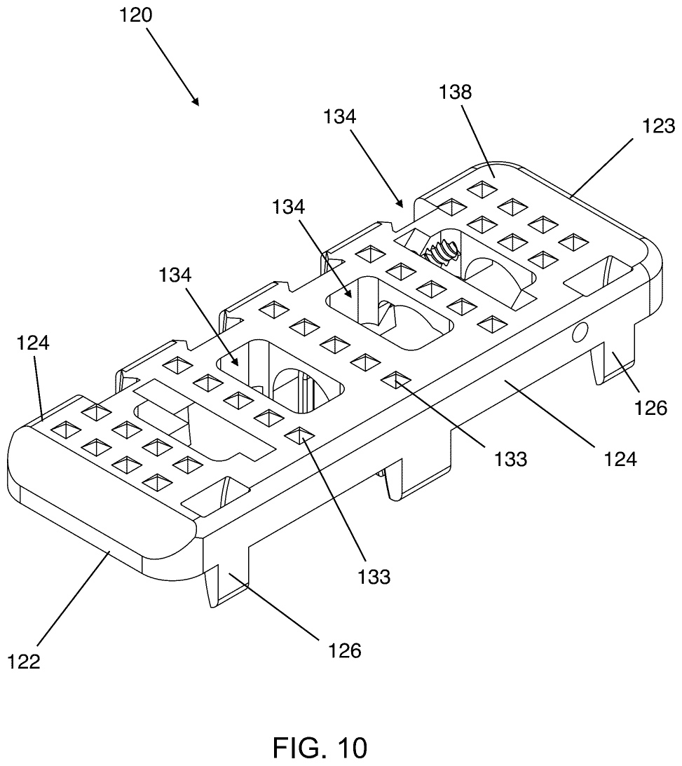

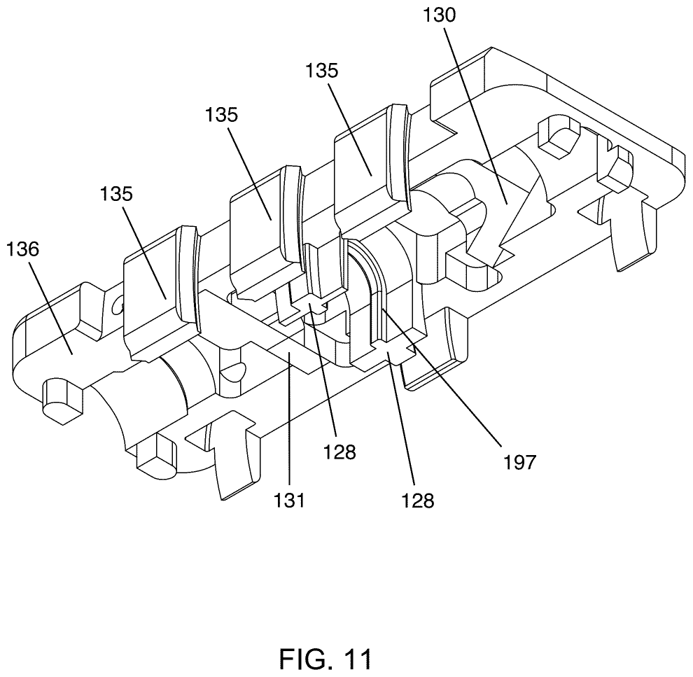

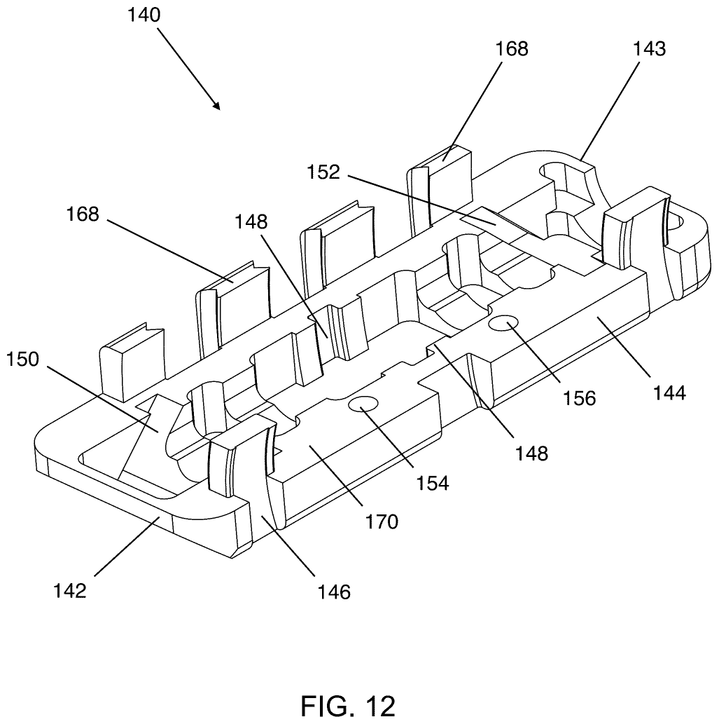

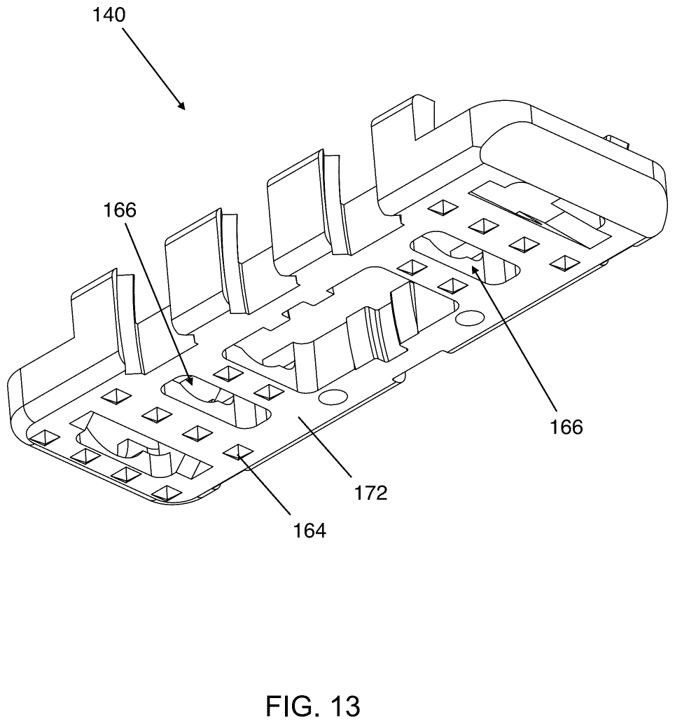

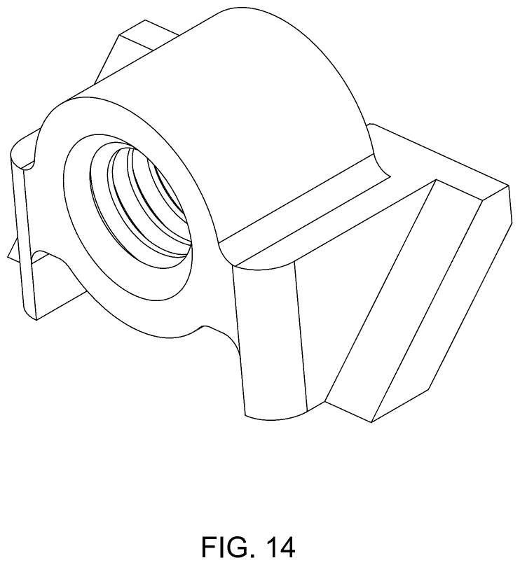

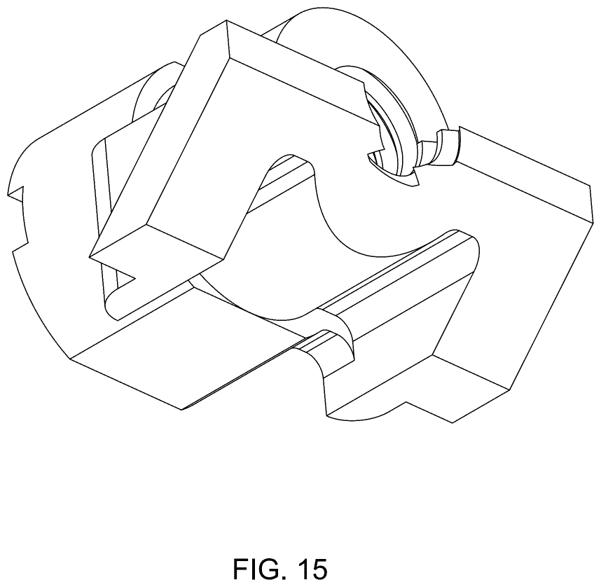

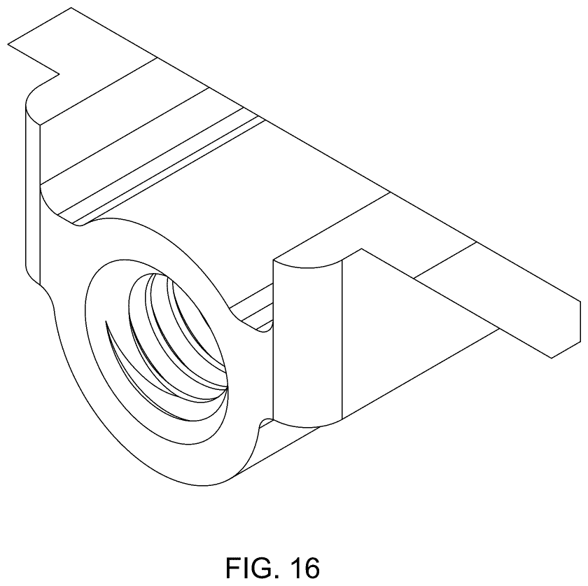

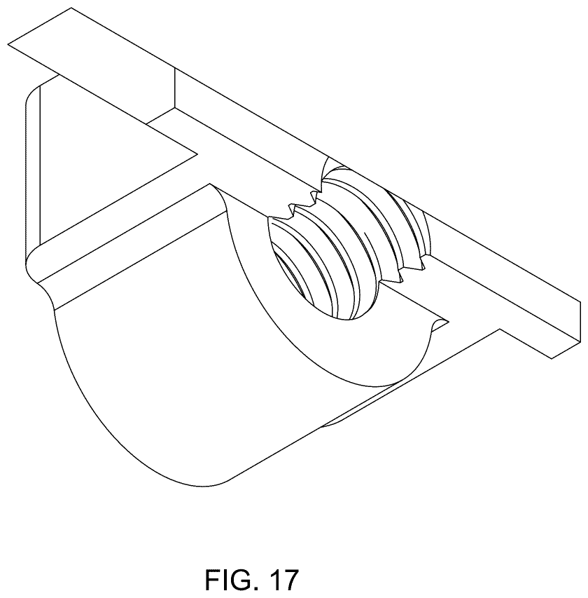

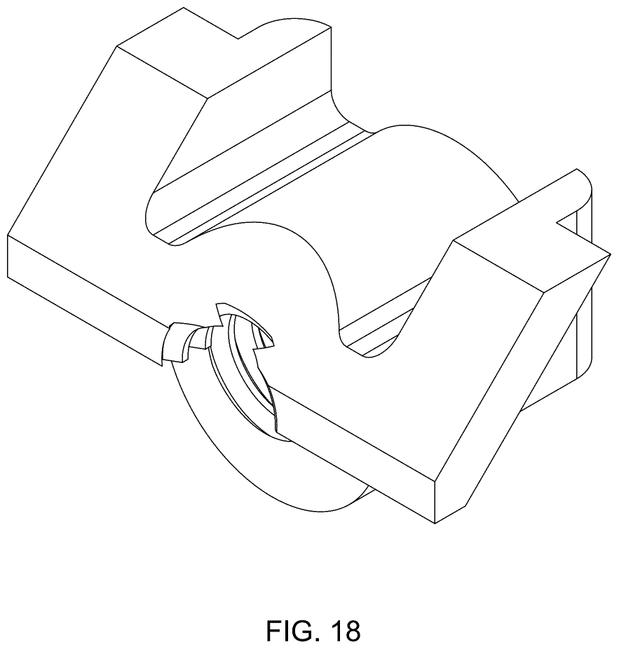

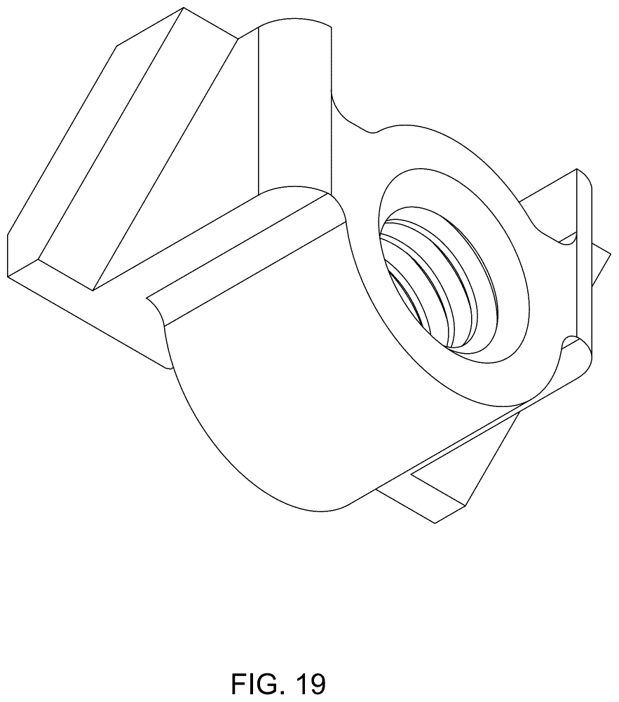

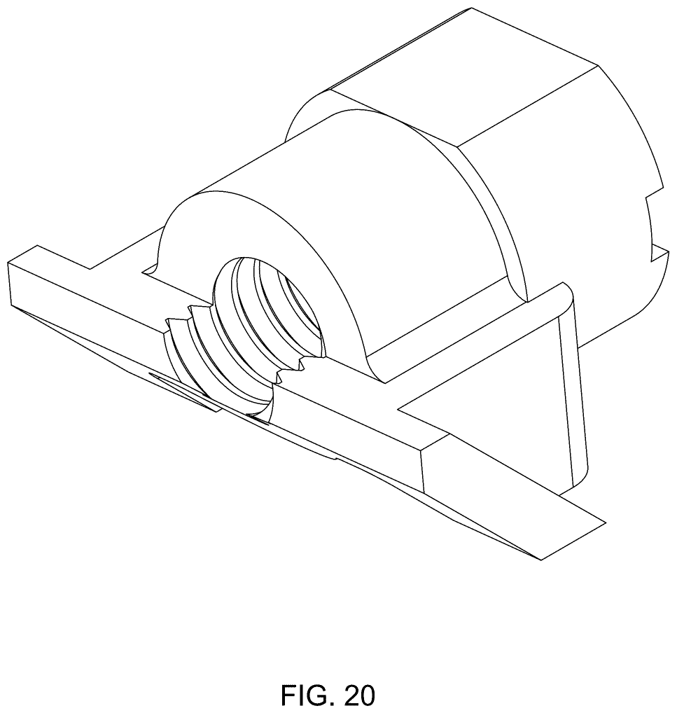

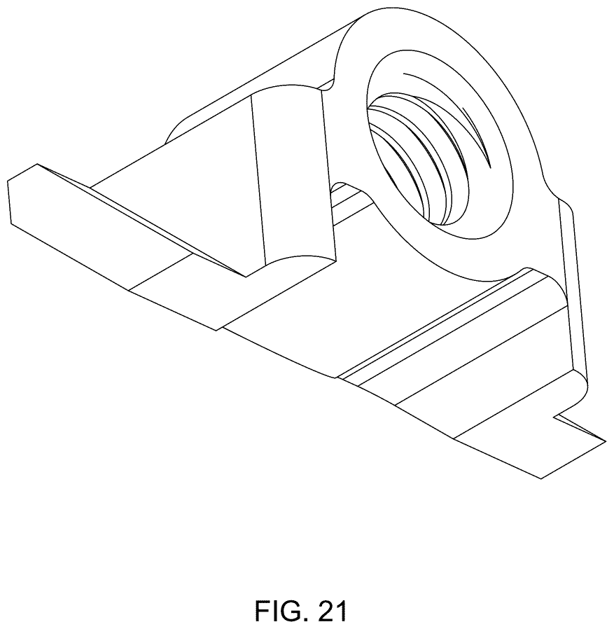

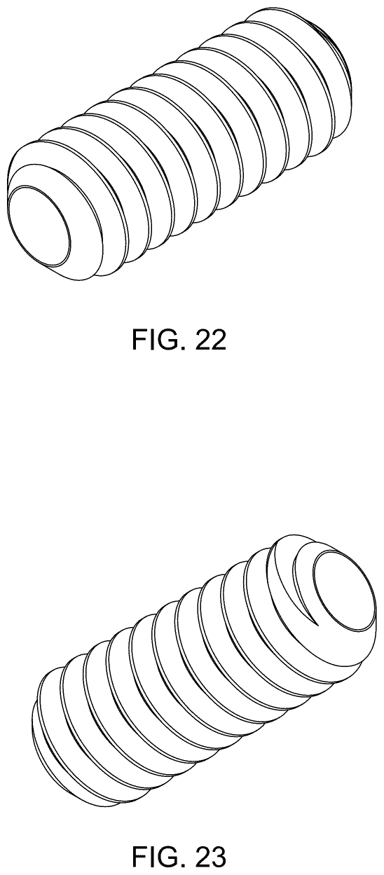

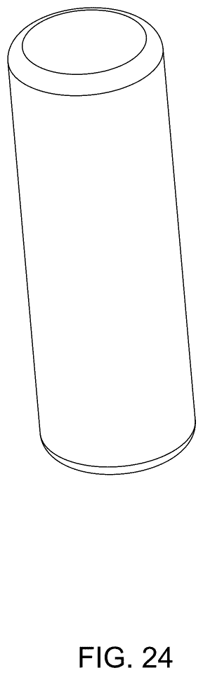

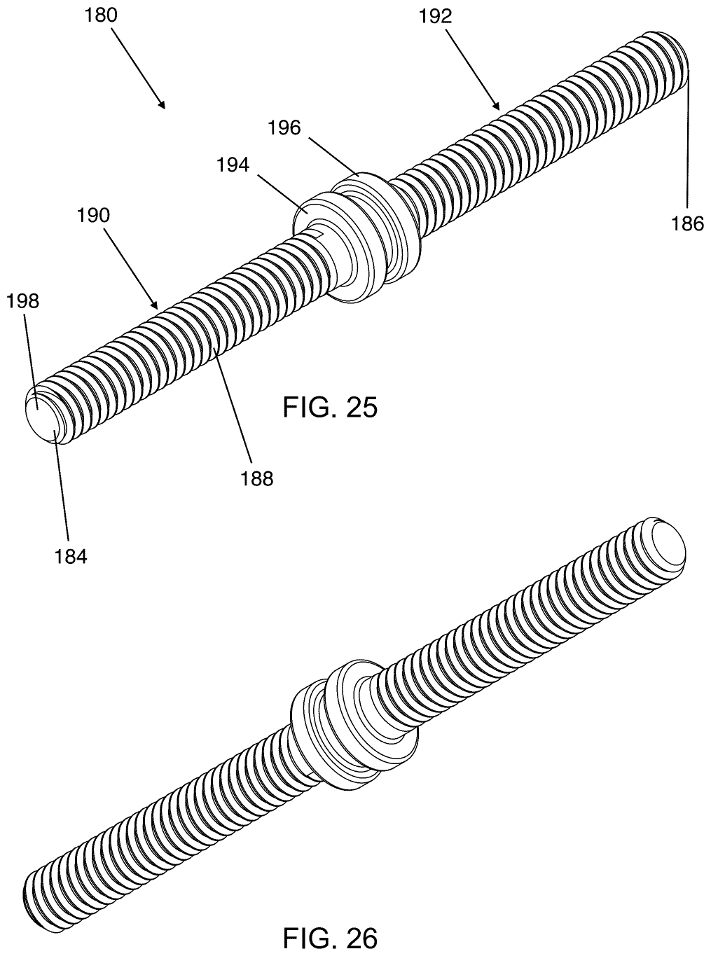

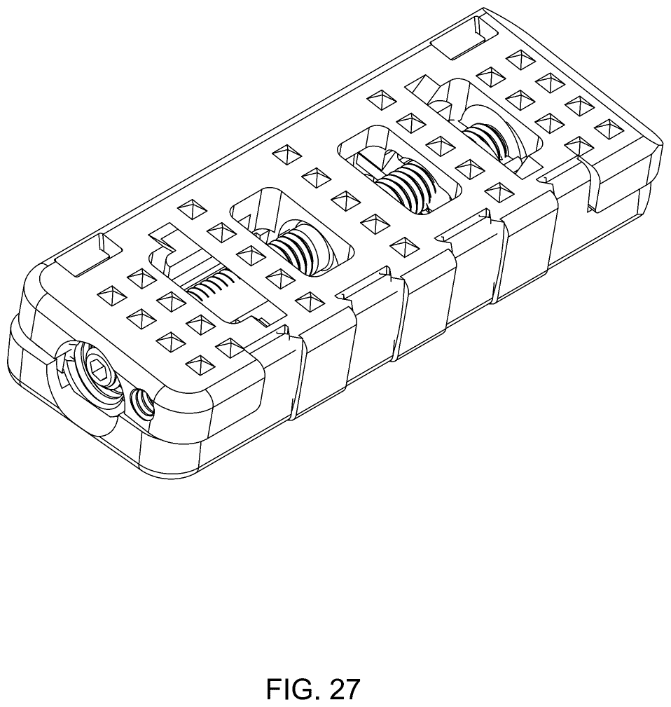

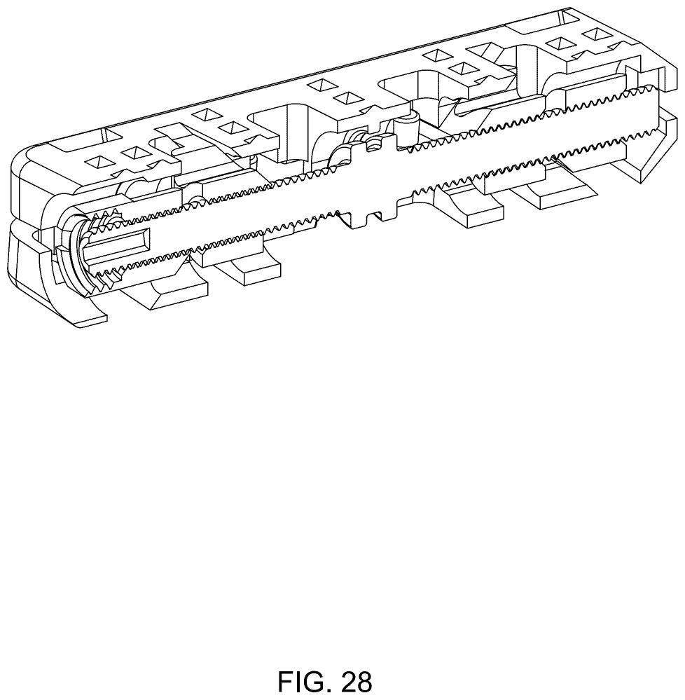

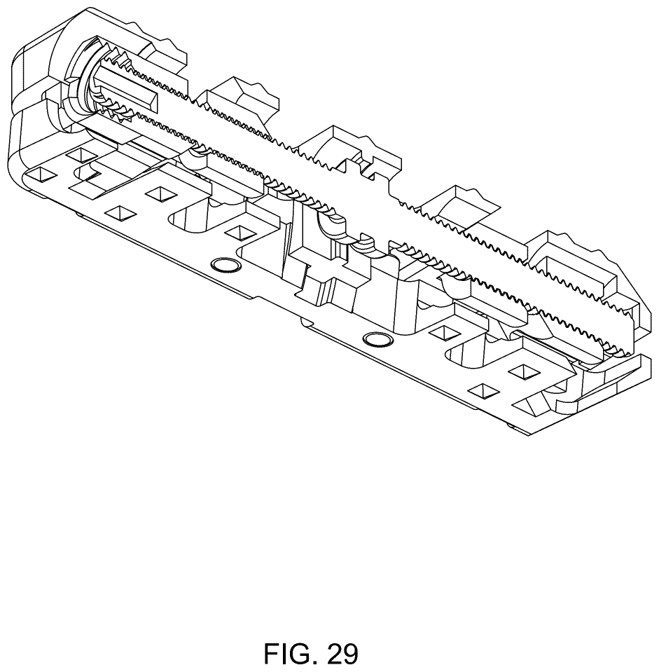

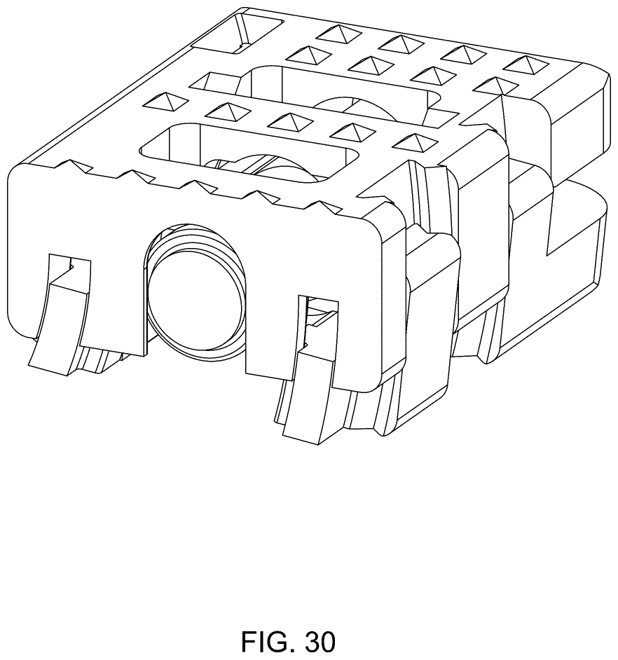

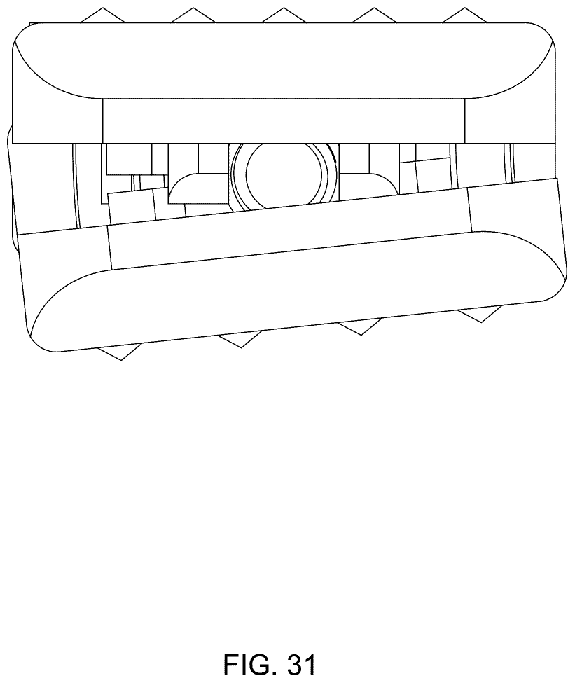

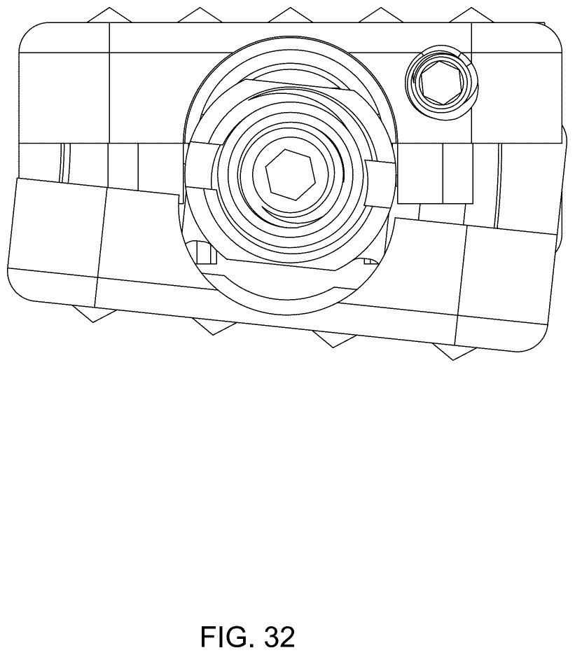

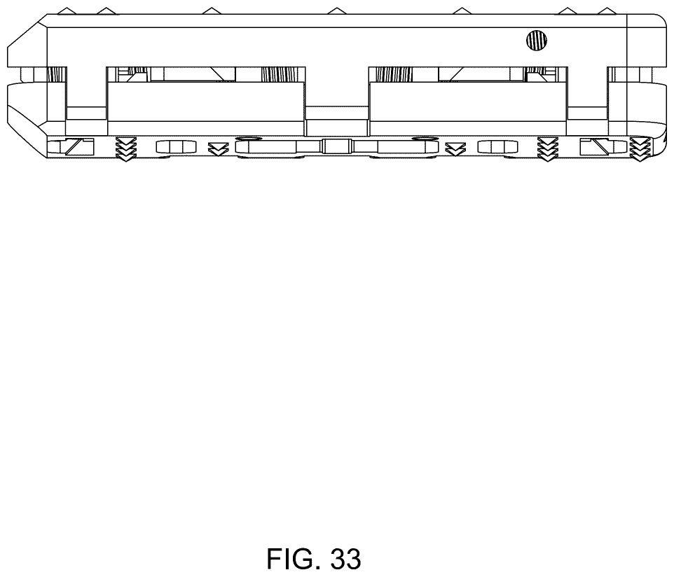

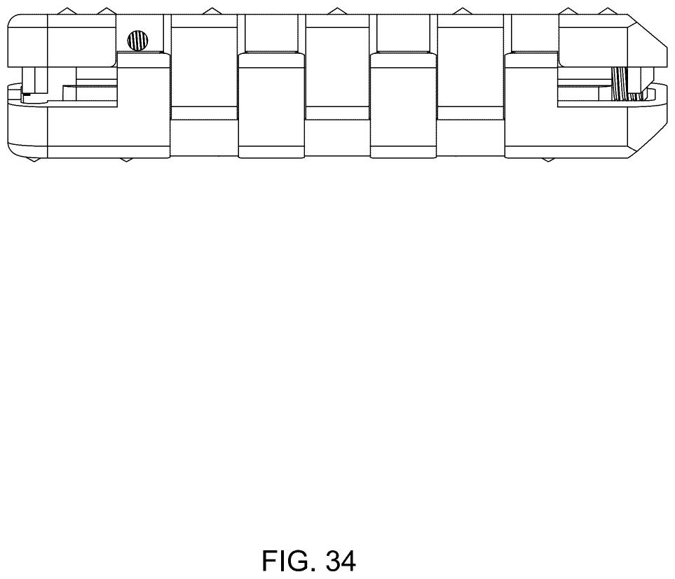

With reference to the figures, and specifically with reference to FIGS. 1 to 26, a lordotic lateral implant, an implant system, process, and method of use are presented. a lordotic lateral implant, an implant system, process, and method of use (hereafter referred to as “lordotic system”, “implant system”, “lordotic implant system”, “alternative embodiment system” or simply “system”) is formed of any suitable size, shape and design.

In the arrangement shown, as one example, system includes a lower plate, an upper plate, a shaft, a right-hand thread, a left-hand thread, a lower plate center, an outer proximal wedge, an outer distal wedge, an inner proximal wedge, an inner distal wedge, a cavity of shaft, a plurality of curved ribs of lower plate, a center curved post of lower plate, a threaded hole of outer proximal wedge, a threaded hole of outer proximal wedge, a linear ramp of outer proximal wedge, a linear ramp of outer distal wedge, a linear ramp of inner proximal wedge, a linear ramp of inner distal wedge, a ramp of upper plate, a ramp of upper plate, a stop of upper plate, a stop of upper plate, stop of upper plate, a secondary feature, a ramp of upper plate, a ramp of lower plate, a threaded boss of outer proximal wedge, a plurality of protusions of lower plate, a plurality of protrusions of upper plate, and a lumen of lower plate, a lumen of upper plate, side posts of the lower plate, side posts of the lower plate, a set screw, a hole, a carved material of the lower plate, a carved material of the upper plate, curved ribs of the lower plate, curved ribs of the upper plate, rails of the lower plate, carved material of, carved material of, carved material, carved material, curved ribs of lower plate, channels, curved ribs of, channels, grooves of, and rib of post.

In the arrangement shown, as one example, system is configured to provide lordotic expansion while maintaining the connection of expansion. Furthermore, system is configured to provide an expandable spacer which expands both opposing sides of the spacer utilizing a single shaft. Furthermore, system is configured to provide a single shaft with both left-handed threading and right-handed threading which offer a number of advantages in the art. Furthermore, system is configured to provide a unique four-wedge design which provides for expansion of the spacer on both sides which provides an optimal and/or customized angle of expansion. Furthermore, system is configured to provide wedges which are symmetrical in two planes. Furthermore, system is configured to provide ramps on both the lower end plate and the upper end plate which have the same slopes. Furthermore, system is configured to provide a shaft which translates when the implant is expanded. Furthermore, system is configured to provide expansion of the spacer and lordotic angle, achieved by the rotation of only a single shaft.

In the arrangement shown, as one example, system is configured with a number of unique features which provide unique functionality not achieved or considered in the state of the art. System is configured to provide wedges which have linear ramps. Furthermore, system is configured with wedges which are in constant contact with the endplate ramps at all times for smooth operation, along with other spacing benefits and advantages in operation. Furthermore, system is configured to provide outer wedges which rotate with respect to inner wedges along the shaft axis. This unique design offers a number of advantages, such as use of a single shaft to provide expansion of both opposing sides of the spacer simultaneously and at a desired ramp angle. Furthermore, and said another way, system is configured to provide lordotic expansion with the rotation of a single shaft. Furthermore, system is configured with a shaft which is not fixed to either the upper or lower plate, providing further advantages and benefits in the art. This is especially important and advantageous considering the state of the art teaches two shafts attached to each opposing side for moving the two sides separately to achieve a goal (such as a desired angle and expansion). Furthermore, and said another way, system is configured to provide a height of expansion on both sides of the implant while expansion occurs. Furthermore, system is configured to provide an over expansion feature. Furthermore, system is configured to provide the state of the art with an anti-collapse feature. These and other benefits and advantages will become more apparent with the description herein and the accompanying figures.

### Configuration

### Upper Plate and Lower Plate:

In the arrangement shown, as one example, system includes a lower plate and an upper plate adjustable coupled and/or operably coupled to the lower plate.

In the arrangement shown, as one example, the lower plate includes a plurality of curved ribs and. The plurality of curved ribs and are configured to provide guidance to the movement and interactions of the upper plate and the lower plate. In the arrangement shown, as one example, the plurality of curved ribs are concentric. Said another way, the movement and/or interaction motion of the upper plate respective to the lower plate is non-linear (in one example). This provides a number of benefits. Linear movement is also hereby contemplated for use.

In the arrangement shown, as one example, the lower plate also includes a curved center post. Curved center post also includes a linear channel which receives the shaft. Said another way, curved center post is designed and configured to receive shaft. The longitudinal displacement of the shaft is restricted by rib of the center post, by this curved center post. The shaft travels linearly along the curved center post while the implant is expanded and/or contracted. Furthermore, the set of side posts and are located in the lower plate (as are further described herein).

### Shaft:

Implant may further include a shaft with a right-hand thread and a left hand thread.

Furthermore, the right hand thread may be on one side of the shaft (nearer a first end) and the left hand thread may be on another side of the shaft (nearer a second end). In this way, the longitudinal movement of the shaft is restricted and/or provided limitations through the lower center plate.

Furthermore, in the arrangement shown, as shown in one example, shaft includes a cavity. In one arrangement, the cavity is configured on the proximal side so as to allow engagement with a tool. The engagement with the tool can cause rotation to the shaft. This tool may be a uniquely configured and shaped tool. Cavity may also be configured to interact with known tooling such as a torx tool, allen type tool (allen wrench), philips shape, and the like.

Furthermore, and in the arrangement shown, as one example, the shaft displaces longitudinally. The longitudinal displacement of the shaft is restricted by this curved center post. The shaft travels along the curved center post while the implant is expanded and/or contracted.

### Wedges:

In the arrangement shown, as one example, system also includes an outer proximal wedge, an outer distal wedge, an inner proximal wedge, and an outer proximal wedge.

In the arrangement shown, as one example, the plurality of wedges are configured in concert with the upper and lower plates such that the wedges are received on the shaft and are movable along the shaft so as to adjust the position of the upper plate and the lower plate. See figures for further illustrations on this operation and movement.

In the arrangement shown, as one example, the outer proximal wedge includes a linear ramp. Similarly, the outer distal wedge includes a linear ramp. The linear ramp of the outer proximal wedge and the linear ramp of the outer distal wedge each have a threaded hole which engages directly with the thread of the shaft. In this way, when the shaft is rotated in an opening direction, the wedges move toward each other, causing the linear ramp to slide against the linear ramp, this causes lordotic expansion. Furthermore, when the shaft is rotated in a closing direction, the wedges move away from one another, causing the linear ramp to slide away from or along linear ramp in a disengaging fashion, which causes lordotic contraction of the implant.

In the arrangement shown, as one example, the inner proximal wedge and the inner distal wedge may be threaded. An unthreaded inner proximal wedge and an unthreaded inner distal wedge are also hereby contemplated for use. Inner wedges move along the shaft.

In the arrangement shown, as one example, outer proximal wedge and outer distal wedge each include linear ramps which engage the upper plate. The inner proximal wedge and the inner distal wedge have linear ramps which engage the lower plate.

Said another way, when the shaft is rotated to expand and/or engage the implant, the outer proximal wedge and the outer distal wedge move and/or slide and/or engage the ramps in a frictional fashion, which forces the upper plate to expand lordotically in a direction away from the lower plate. Similarly, when the shaft is rotate to expand the implant, the inner proximal wedge and the inner distal wedge ride along, slide along, and or frictionally force the ramps of the lower plate. In this way, the outer and inner wedges move in the opposite direction relative to each other on the shaft. Furthermore, when the implant is expanded and/or in the expanded position, the height on one side and/or the distance from the lower plate to the upper plate on one of the opposing sides is larger than the distance from the upper plate to the lower plate of the second of the opposing sides and/or of the opposite side of the opposing sides. In this way, the implant acts uniquely as a lordotic implant and provides simultaneous expansion using a single shaft but creating different distances on the opposing sides of the implant.

### Over Expansion and Other Features:

Over expansion and/or a particular distance of expansion can be predetermined prior to installation of the implant. System is configured to prevent over expansion with the inclusion of a sleeve around the shaft which stops expansion at a particular preset, depending on the needs of the patient. In another way, a stop can be installed which prevents the expansion past a particular distance. This can be achieved by inclusion of a stop on the upper plate. In the arrangement shown, as one example, stops are shown on one plate for ease of explanation. However, stops can be located on either the upper plate or the lower plate or both.

After an implant has been installed, the stabilization of that implant is important. For longevity and effectiveness, implant includes a locking feature of the shaft (locking in concert with the outer proximal wedge) utilizing a secondary feature so as to lock the implant at the desired expansion level.

In the arrangement shown, as one example, the upper plate and lower plate include ramps. Ramps are configured on the distal side. These ramps are configured to allow easier insertion and less invasive implanting into the vertebral disc space. Similarly, and said another way, the outer proximal wedge includes and is configured to have a threaded boss on the proximal side which provides for engagement with an inserter. This aids in insertion of the implant. This also causes installation to be less invasive than current methods in the state of the art. Additionally, implant includes a plurality of protrusions which provide for improved engagement with the end plates of a vertebrae and/or bone surface and/or engagement surface. Furthermore, and in the arrangement shown, system includes a lower plate and an upper plate each having a lumen which is configured to accept a bone graft.

### Side Posts:

In the arrangement shown, as one example, system also includes side posts of the lower plate and side posts of the lower plate. The sets of side posts are located in the lower plater and prevent the shaft from moving, shifting and/or rocking from side to side. Said another way the side posts prevent unwanted motion of the shaft, and in particular rocking or shifting.

### Set Screw and Hole:

In the arrangement shown, as one example, system also includes a set screw. Set screw is sized, shaped, and designed to prevent the implant from collapsing by engaging. The set screw is located in a threaded hole in one of the endplates. The set screw stops the translation of one of the internal wedges. In this way, the set screw maintains an expanded implant.

Furthermore, the plate which holds the set screw also includes a hole perpendicular to the screw hole. In this way, when the set screw is engaged and/or in an engaged position, the hole is covered. In this way, the hole (being covered) conveys to the surgeon under fluoro that the implant is locked and secured in place. If the hole is visible to the surgeon, then the surgeon has not locked the implant into place.

### Carved Material and Channels:

In the arrangement shown, as one example, system also includes carved material and of the lower plate and the upper plate. Furthermore, the wedges also includes carved material. The carved material on the endplates and the wedges creates the channels. Channels are configured to provide the flow of bone graft from the proximal side of the distal side of the implant.

### Curved Ribs and Rails:

In the arrangement shown, as one example, system also includes curved ribs, and rails. Curved ribs and rails are located in the lower plate and are configured to guide the movement of the upper plate. The curved ribs and rails are concentric. Said another way, curved ribs and rails guide the upper plate. The movement of upper plate is non-linear with respect to lower plate.

### Grooves and Rib:

In the arrangement shown, as one example, system includes curved ribs, which are located in the upper plate and are configured, designed, and shaped to guide the movement of the lower plate. All the curves of the ribs are concentric. The ribs also include grooves which engage with the rails. In this way, the grooves and rails guide the upper plate.

### Additional Features:

In addition to the features and components disclosed herein, an anti-collapse feature is hereby contemplated for use. Anti-collapse feature is configured to prevent the collapse of system 10 and provide stability therein.

In addition to the features and components disclosed herein, a lock and/or unlock visibility feature is contemplated. Lock and/or unlock visibility feature provides an indicator that the system is locked in place and/or unlocked (allowing for expansion and/or contraction).

### User (or Plurality Thereof):

In the arrangement shown, as one example, system may include a user. User may be any user interacting with or utilizing the system. This may include viewing, controlling, analyzing, manipulating, and/or interacting with system. User is not limited to a single user but may be a plurality of users.

In addition to the above identified features, options, controls, and components, system 100 may also include other features and functionalities, among other options, controls, and components.

It will be appreciated by those skilled in the art that other various modifications could be made to the system, process, and method of use without parting from the spirit and scope of this disclosure. All such modifications and changes fall within the scope of the claims and are intended to be covered thereby.

1. A lordotic lateral implant, comprising:
a lower plate;
the lower plate extending a length from a first end to a second end between opposing sides;
the lower plate having a plurality of curved ribs;
the lower plate having a center post;
the lower plate having a first ramp;
the lower plate having a second ramp;
the lower plate having a plurality of protrusions;
the lower plate having an interior surface;
the lower plate having an exterior surface;
an upper plate;
the upper plate extending a length from a first end to a second between opposing sides;
the upper plate having a plurality of curved rib apertures;
wherein the plurality of curved rib apertures accept the plurality of curved ribs of the lower plate;
the upper plate having a center post aperture for accepting the center post of the lower plate;
the upper plate having a first ramp;
the upper plate having a second ramp;
the upper plate having a plurality of protrusions;
the upper plate having an interior surface;
the upper plate having an exterior surface;
a shaft;
the shaft extending a length from a first end to a second end;
the shaft having an outer surface;
the shaft having a first threading;
the shaft having a second threading;
the shaft having a first guide;
the shaft having a second guide;
an outer proximal wedge;
an inner proximal wedge;
wherein rotating the shaft causes expansion of the lordotic lateral implant; wherein expansion of the lordotic lateral implant is caused when the shaft is rotated, causing the outer proximal wedge and the inner proximal wedge to move along the first ramp and the second ramp, increasing the volume between the upper plate and the lower plate.

2. The implant of claim 1, further comprising:
wherein the first threading is a right handed threading.

3. The implant of claim 1, further comprising:
wherein the second threading is a left handed threading.

4. The implant of claim 1, further comprising:
wherein rotation of the shaft causes equal expansion across the entire plane of the upper plate and the lower plate.

5. The implant of claim 1, further comprising:
the lower plate having a third ramp;
the upper plate having a third ramp;
wherein the third ramp of the upper plate and the third ramp of the lower plate have the same slope.

6. The implant of claim 1, further comprising:
the lower plate having a lumen;
the upper plate having a lumen.

7. The implant of claim 1, further comprising:
the lower plate having a plurality of attachment features;
the upper plate having a plurality of attachment features.

8. The implant of claim 1, further comprising:
the upper plate having a first stop;
the upper plate having a second stop;
the upper plate having a third stop;
the upper plate having a fourth stop.

9. The implant of claim 1, further comprising:
wherein the first guard and the second guard engage a ridge of the center post; the center post preventing movement laterally of the shaft;
wherein the center post is straight on an interior side of the center post;
wherein the center post is curved on an exterior side of the center post.

10. The implant of claim 1, further comprising:
the shaft having an aperture adjustment;
the shaft having an adjustment connection feature.

11. The implant of claim 1, further comprising:
an outer distal wedge;
an inner distal wedge.

12. The implant of claim 1, further comprising:
wherein the expansion of the lordotic lateral implant occurs while maintaining the connection of expansion; wherein the connection of expansion is a connection between the upper plate and the lower plate such that the lower plate and upper plate maintain stability as expansion of the lordotic lateral implant occurs.

13. The implant of claim 1, further comprising:
wherein the expansion of the lordotic lateral implant can vary depending on need; the expansion of the lordotic implant varying based on the amount of rotation of the shaft.

14. The implant of claim 1, further comprising:
wherein rotating the shaft causes movement of the outer proximal wedge and the inner proximal wedge.

15. The implant of claim 1, further comprising:
wherein rotating the shaft causes movement of the outer distal wedge and the inner distal wedge.

16. The implant of claim 1, further comprising:
wherein the wedges are symmetrical in two planes.

17. The implant of claim 1, further comprising:
wherein movement of the outer proximal wedge and the inner proximal wedge causes either expansion or contraction of the lordotic lateral implant; wherein contraction of the lordotic lateral implant is causing a decrease in volume between the upper plate and the lower plate.

18. The implant of claim 1, further comprising:
wherein the first ramp of the upper plate and the first ramp of the lower plate have the same slope;
wherein the second ramp of the upper plate and the second ramp of the lower plate have the same slope.

19. The implant of claim 1, further comprising:
a set screw;
wherein the set screw is configured to engage and disengage;
wherein the set screw is configured to engage by tightening and preventing the translation of a plurality of wedges;
wherein the set screw is configured to disengage by loosening and allowing the translation of the plurality of wedges;
wherein when the set screw is engaged the implant is prevents from expanding or contracting;
wherein when the set screw is disengaged the implant is free to expand and contract.

20. The implant of claim 19, further comprising:
a lock/unlock indicator feature;
the lock/unlock indicator feature provides a clear view of the set screw;
wherein when the set screw is in an engaged position, the view of the lock/unlock indicator feature shows the set screw is in the engaged position as the lock/unlock indicator feature shows the set screw in place;
wherein when the set screw is in a disengaged position, the view of the lock/unlock indicator feature shows a clear view into the interior of the implant.

21. A lordotic implant system, comprising:
a hand tool;
a lower plate;
the lower plate extending a length from a first end to a second end between opposing sides;
the lower plate having a plurality of curved ribs;
the lower plate having a center post;
the lower plate having a first ramp;
the lower plate having a second ramp;
the lower plate having a third ramp;
the lower plate having a plurality of protrusions;
the lower plate having a lumen;
the lower plate having a plurality of attachment features;
the lower plate having an interior surface;
the lower plate having an exterior surface;
an upper plate;
the upper plate extending a length from a first end to a second between opposing sides;
the upper plate having a plurality of curved rib apertures;
wherein the plurality of curved rib apertures accept the plurality of curved ribs of the lower plate;
the upper plate having a center post aperture for accepting the center post of the lower plate;
the upper plate having a first ramp;
the upper plate having a second ramp;
the upper plate having a third ramp;
the upper plate having a first stop;
the upper plate having a second stop;
the upper plate having a third stop;
the upper plate having a fourth stop;
the upper plate having a plurality of protrusions;
the upper plate having a lumen;
the upper plate having a plurality of attachment features;
the upper plate having an interior surface;
the upper plate having an exterior surface;
a shaft;
the shaft extending a length from a first end to a second end;
the shaft having a cavity; wherein the cavity accepts the hand tool;
the shaft having an outer surface;
the shaft having a first threading;
the shaft having a second threading;
the shaft having a first guide;
the shaft having a second guide;
the shaft having an aperture adjustment;
the shaft having an adjustment connection feature;
an outer proximal wedge;
an inner proximal wedge;
an outer distal wedge;
an inner distal wedge;
wherein the hand tool is used to cause rotation of the shaft;
wherein rotating the shaft causes expansion of the lordotic lateral implant; wherein expansion of the lordotic lateral implant is cause the upper plate and lower plate to create an increased volume between the upper plate and the lower plate;
wherein the expansion of the lordotic lateral implant occurs while maintaining the connection of expansion; wherein the connection of expansion is a connection between the upper plate and the lower plate such that the lower plate and upper plate maintain stability as expansion of the lordotic lateral implant occurs;
wherein the expansion of the lordotic lateral implant can vary depending on need; the expansion of the lordotic implant varying based on the amount of rotation of the shaft;
wherein rotating the shaft causes movement of the outer proximal wedge and the inner proximal wedge;
wherein rotating the shaft causes movement of the outer distal wedge and the inner distal wedge;
wherein movement of the outer proximal wedge and the inner proximal wedge causes either expansion or contraction of the lordotic lateral implant; wherein contraction of the lordotic lateral implant is causing a decrease in volume between the upper plate and the lower plate;
wherein the wedges are symmetrical in two planes;
wherein the first ramp of the upper plate and the first ramp of the lower plate have the same slope;
wherein the second ramp of the upper plate and the second ramp of the lower plate have the same slope;
wherein the third ramp of the upper plate and the third ramp of the lower plate have the same slope.

22. A method of expansion of a lordotic implant with rotation of a single shaft, comprising the steps:
providing the lordotic implant, the lordotic implant including:
a lower plate;
the lower plate extending a length from a first end to a second end between opposing sides;
the lower plate having a plurality of curved ribs;
the lower plate having a center post;
the lower plate having a first ramp;
the lower plate having a second ramp;
the lower plate having a third ramp;
the lower plate having a plurality of protrusions;
the lower plate having a lumen;
the lower plate having a plurality of attachment features;
the lower plate having an interior surface;
the lower plate having an exterior surface;
an upper plate;
the upper plate extending a length from a first end to a second between opposing sides;
the upper plate having a plurality of curved rib apertures;
wherein the plurality of curved rib apertures accept the plurality of curved ribs of the lower plate;
the upper plate having a center post aperture for accepting the center post of the lower plate;
the upper plate having a first ramp;
the upper plate having a second ramp;
the upper plate having a third ramp;
the upper plate having a first stop;
the upper plate having a second stop;
the upper plate having a third stop;
the upper plate having a fourth stop;
the upper plate having a plurality of protrusions;
the upper plate having a lumen;
the upper plate having a plurality of attachment features;
the upper plate having an interior surface;
the upper plate having an exterior surface;
a shaft;
the shaft extending a length from a first end to a second end;
the shaft having a cavity; wherein the cavity accepts the hand tool;
the shaft having an outer surface;
the shaft having a first threading;
the shaft having a second threading;
the shaft having a first guide;
the shaft having a second guide;
the shaft having an aperture adjustment;
the shaft having an adjustment connection feature;
an outer proximal wedge;
an inner proximal wedge;
an outer distal wedge;
an inner distal wedge;
providing a hand tool;
inserting the implant into position;
inserting the hand tool into the aperture adjustment of the shaft;
causing the shaft to rotate by the hand tool
wherein the hand tool is used to cause rotation of the shaft;
wherein rotating the shaft causes expansion of the lordotic lateral implant; wherein expansion of the lordotic lateral implant is cause the upper plate and lower plate to create an increased volume between the upper plate and the lower plate;
wherein the expansion of the lordotic lateral implant occurs while maintaining the connection of expansion; wherein the connection of expansion is a connection between the upper plate and the lower plate such that the lower plate and upper plate maintain stability as expansion of the lordotic lateral implant occurs;
wherein the expansion of the lordotic lateral implant can vary depending on need; the expansion of the lordotic implant varying based on the amount of rotation of the shaft;
wherein rotating the shaft causes movement of the outer proximal wedge and the inner proximal wedge;
wherein rotating the shaft causes movement of the outer distal wedge and the inner distal wedge;
wherein movement of the outer proximal wedge and the inner proximal wedge causes either expansion or contraction of the lordotic lateral implant; wherein contraction of the lordotic lateral implant is causing a decrease in volume between the upper plate and the lower plate;
wherein the wedges are symmetrical in two planes;
wherein the first ramp of the upper plate and the first ramp of the lower plate have the same slope;
wherein the second ramp of the upper plate and the second ramp of the lower plate have the same slope;
wherein the third ramp of the upper plate and the third ramp of the lower plate have the same slope.
